(19) United States
(12) Patent Application Publication
(10) Pub. No.: US 2018/0303830 A1
(43) Pub. Date: Oct. 25, 2018

(54) CANCERS EXPRESSING CCR5 AND METHODS OF TREATMENT OF SAME

(71) Applicant: THOMAS JEFFERSON UNIVERSITY, Philadelphia, PA (US)

(72) Inventors: Richard Pestell, Philadelphia, PA (US); Massimo Cristofanilli, Philadelphia, PA (US); Xuanmao Jiao, Rockville, MD (US)

(73) Assignee: THOMAS JEFFERSON UNIVERSITY, PHILADELPHIA, PA (US)

(21) Appl. No.: 15/738,020

(22) PCT Filed: Jun. 22, 2016
(86) PCT No.: PCT/US16/38713

§ 371 Date: Dec. 19, 2017

Related U.S. Application Data
(60) Provisional application No. 62/183,058, filed on Jun. 22, 2015.

(51) Int. Cl. A61K 31/506; A61K 31/46; A61P 35/00; G01N 33/574; G01N 33/566

(52) U.S. Cl. CPC A61K 31/506; A61K 31/46; A61P 35/00; A61K 45/06; G01N 33/566; A61K 2300/00; G01N 33/57434

(57) ABSTRACT

Embodiments disclosed herein provide for methods of treating cancer and detecting CCR5 on circulating tumor cells and uses thereof based upon the same.

### PRIORITY CLAIM

This application claims the benefit of U.S. Provisional Application Ser. No. 62/183,058, filed Jun. 22, 2015, the disclosure content of which is hereby incorporated by reference in its entirety.

### FIELD OF INVENTION

The present application is generally related to cancers expressing CCR5 and methods of determining candidates for treatment of certain cancer forms that express CCR5 and methods of treating the same.

### BACKGROUND OF THE INVENTION

Breast cancer contributes to the death of over 400,000 women in the world, and more than 40,000 women in the United States annually (26). In 20-30% of patients, the relapse of patients with metastatic breast cancer contributes the main cause of death (27). The basal breast cancer genetic subtype is associated with increased risk of metastasis and reduced survival rates compared with either luminal A or B tumors (28, 29). As basal breast tumors are typically deficient in expression of Her2, the androgen receptor (AR) estrogen receptor (ERα), patients with basal breast cancer subtype are considered unlikely to respond to hormonal or Her2 targeted therapeutic intervention. Recent studies demonstrated that the G protein-coupled receptor family (GPCR) member CCR5 is over-expressed in basal breast cancer (1).

Chemokines binding to GPCR mediate distinct biological processes in immune surveillance and in tumorigenesis. The chemokine receptors CXCR4 and the ligand CCR7 are known to be expressed in breast cancer cells and in metastatic human breast cancer. The CCR5 ligand CCL5 (RANTES) correlates with disease progression in patients with breast cancer (30, 31). Oncogenic transformation of immortal human breast cancer (MCF10A) cells with a single oncogene (either Ha-Ras, RAS, c-Myc, v-Src, or ErbB2) is sufficient for the induction of CCR5 expression (1).

Interrogation of microarray databases of 2,254 human breast cancers demonstrated that CCL5/CCR5 signaling is activated in the basal and Her2 breast cancer subtypes (1). Furthermore, CCR5+ vs. CCR5− from within the same breast cancer contributed to invasiveness and breast cancer metastasis. The CCR5 antagonist Maraviroc (Selzentry) is approved for use in CCR5 trophic HIV, whereas Vicriviroc (SCH417690) demonstrated safety and partial therapeutic responses.

Recent studies have demonstrated a propensity of tumor initiating cells with stem cell-like features to contribute to metastasis and therapy resistance (9, 32). The mechanisms by which cancer stem cells survive chemotherapy- and radiotherapy-induced death correlates with mechanisms predicting genomic integrity (33). Heterogeneous DNA-damaging agents contribute distinct genetic lesions, which are repaired through distinct but frequently overlapping mechanisms. The basic excision repair (BER) system targets small chemical alterations (base modifications) and includes PCNA and LIG3 (ligase 3 DNA ATP-dependent polymerase) (DNA-directed). The homologous recombination repair (HRR) is the predominant repair system in dividing cells, and together with the non-homologous enjoining (NHEJ), contributes the majority of chemotherapy- and radiotherapy-induced DNA damage repair. HRR proteins include XRCC2 (x-ray repair complementing defective repair in Chinese hamster cells 2). The Fanconi anemia pathway mediates intra-strand DNA cross links and is mutated in the Fanconi anemia disease. Fanconi anemia is associated with predisposition to leukemia and cancer, including breast and ovarian cancer and components of the Fanconi anemia pathway are mutated in human breast cancer (34, 35). The HRR is associated with hereditary breast and ovarian cancer syndromes. Defective BER contributes to cancer predisposition. The current studies examined the role of CCR5 populations within basal breast cancer and demonstrated the enhanced propensity towards cancer stem cell formation and activation of DNA repair mechanisms by CCR5.

Indeed, treatment for metastatic cancer is inadequate, reflected the large number of deaths from metastatic cancer in the world each year. Cancer therapies often provide targeted approaches to damage the DNA of cancerous cells. However, several forms of cancer have been found to have poor response rates to these forms of cancer therapies, and new, targeted therapies are needed to combat these aggressive and deadly cancers.

Currently, the largest targeted therapy is HER2 and Herceptin. HER2 is expressed in large number of cells, including normal and cancer cells and is overexpressed in 17% of breast cancers. However, HER2 is also expressed in many normal cells; therefore, targeted treatments often have a modest therapeutic response.

CCR5 as a comparison is expressed in vast majority of malignant cells, not normal cells, like HER2. CCR5 is only expressed in a small subset of immune cells. CCR5 has been found to be selectively overexpressed in a subset of malignancies, including in approximately 50% of breast, prostate, and other malignancies.

A published study of over 2,200 breast cancer patients demonstrated increased expression of CCR5 in a substantial proportion of patients, primarily in the basal breast cancer subtype (1). Basal breast cancer has a poor prognosis and the patients' tumors typically do not express other therapeutic targets such as ErbB2, ER or AR. The data demonstrated that oncogenic transformation of immortal human breast cancer cells, with either Ha-Ras, c-Myc, ErbB2 (NeuT) or c-Src, induces the mRNA expression and protein abundance of CCR5 during the process of transformation (1). See also, For the Diagnosis, Prognosis and Treatment of Cancer Related Applications—PCT/US2012/028546.

In a study of prostate cancer (2), it was demonstrated that in a similar manner to breast cancer, oncogenic transformation of primary prostate epithelial cells (with Ha-Ras, c-Myc, ErbB2 (NeuT) or v-Src), is sufficient to induce CCR5 expression, and the invasive phenotype. The study demonstrated that metastasis of oncogene transformed prostate cancer cells to the bones and brain of mice was blocked by CCR5 inhibitors at the same dose as used in humans for HIV treatment.

### SUMMARY OF THE INVENTION

In accordance with these and other objects, some embodiments disclosed herein are directed to methods for measuring CCR5 expressed on circulating tumor cells in the blood of a patient suffering from cancers expressing CCR5.

In some embodiments, methods of detecting CCR5 expressing transformed cells in circulating tumor cells (CTC) in the blood of humans; wherein the detection of said cells is used to monitor cancer treatment that targets CCR5 are provided.

In some embodiments, methods for monitoring treatment of a patient who has been administered a CCR5 inhibitor comprising measuring CCR5 expression in circulating cancer cells of the patient are provided.

In some embodiments, methods for determining therapeutic substratification comprising administering to a patient an effective amount of a CCR5 inhibitor and subsequent to the administration of said CCR5 inhibitor, measuring the presence of CCR5 on the patient's primary breast tumor by measuring the CCR5 on circulating tumor cells are provided.

In some embodiments, methods for treating a cancer expressing CCR5 comprising administering to a patient an effective amount of a CCR5 antagonist are provided.

In some embodiments, methods of treating a cancer expressing CCR5 comprising administering to a patient an effective amount of a CCR5 inhibitor and concomitantly administering to said patient a treatment effective to modify cell DNA are provided.

In some embodiments, methods of treating a cancer expressing CCR5 comprising administering to a patient an effective amount of a CCR5 inhibitor and concomitantly administering to said patient a chemotherapeutic, radiation therapy, or PARP inhibitor are provided.

In some embodiments, methods of treating a cancer expressing CCR5 comprising administering to a patient an effective amount of a CCR5 inhibitor and concomitantly administering to said patient a treatment effective to damage DNA of cancer cells are provided.

In some embodiments, methods for reducing DNA cancer cell repair comprising administering to a patient an effective amount of a CCR5 inhibitor that reduces the ability of CCR5 to repair damaged cancer cells are provided.

In some embodiments, methods for improving the efficacy of a therapeutic treatment comprising administering a CCR5 inhibitor to a patient and providing a further administration to said patient of a second therapeutic treatment targeting cancerous cells are provided.

In some embodiments, methods for detecting candidate patients for CCR5-based therapies comprising detecting CCR5 expression in CTC cells, wherein detection of CCR5 expression provides for treatment with a CCR5 inhibitor or antagonist to overcome the chemotherapy and radiation therapy resistance are provided.

In some embodiments, methods od for reducing the amount of a cancer therapeutic administered to a patient receiving therapeutic treatment selected from the group consisting of chemotherapy, brachytherapy, and a PARP inhibitor; wherein the dose of the cancer therapeutic is reduced from a prior dose by administering to said patient an effective amount of a CCR5 antagonist and then administering to said patient the reduced amount of the cancer therapeutic.

In some embodiments, methods of treating a subject with cancer, the method comprising detecting the presence of CCR5 on circulating tumor cells in a subject's sample; and administering to the subject with CCR5 on the circulating tumors cells a CCR5 inhibitor to treat the cancer are provided.

In some embodiments, methods of identifying and treating a cancer in a subject as susceptible to a CCR5 inhibitor are provided. In some embodiments, the method comprises obtaining a sample from the subject; detecting the presence of CCR5 on circulating tumor cells in the subject's sample; identifying the cancer as susceptible to a CCR5 inhibitor when CCR5 is found to be present on the circulating tumor cells; and administering to the identified subject a CCR5 inhibitor to treat the cancer.

In some embodiments, methods of treating a subject with cancer, the method comprising detecting the presence of CCR5 on circulating tumor cells in a subject's sample; and administering to the subject with CCR5 on the circulating tumors cells a CCR5 inhibitor and a DNA damaging agent to treat the cancer are provided.

In some embodiments, methods of detecting cancer in a subject are provided, wherein the methods comprise detecting CCR5 expression on circulating tumor cells in a blood sample obtained from the subject, wherein the cancer is detected when CCR5 expression is detected on the circulating tumor cells in the blood sample obtained from the subject.

In some embodiments, methods of treating a chemotherapeutic resistant or radiation resistant tumor in a subject are provided, wherein the methods comprise administering to the subject a CCR5 inhibitor.

In some embodiments, methods of monitoring the effectiveness of a treatment on CCR5 positive cancer are provided, wherein the methods comprise administering a cancer treatment to a subject with a CCR5 positive cancer; detecting CCR5 expression on circulating tumor cells in a sample obtained from the treated subject, wherein a decrease in CCR5 expression on the circulating tumor cells as compared to a sample from the subject before treatment indicates that the treatment is effective on the CCR5 positive cancer.

### BRIEF DESCRIPTION OF THE DRAWINGS

FIG. 1 depicts images of SUM149 example cells stained with CCR5 (red) or DAPI (blue)

FIG. 2 depicts images of inflammatory breast cancer (IBC-02) stained for CCR5, cytokeratin, DAPI and merged cells.

FIGS. 3A, 3B, 3C, and 3D depict images of cells and a graphical representation of Maraviroc and Vicriviroc as compared to a control at controlling migration and invasion across the noncoated membrane and matrigel-coated membrane.

FIGS. 4A, 4B, 4C, and 4D depict CCR5⁺ population of SUM159 cells are enriched with breast cancer stem cells which showed by mammosphere formation assay.

FIGS. 5A, 5B, and 5C depict CCR5⁺ SUM159 cells are more metastatic to the lungs of mice than CCR5⁻ SUM159 cells.

FIGS. 6A, 6B, and 6C depict overexpression of CCR5 in SUM159 breast cancer cells enhance their tumorigenecity.

FIGS. 7A and 7B depict endogenous CCR5 promotes lung metastases by showing invasion of CCR5 activity by maraviroc reduces in mice.

FIGS. 8A, 8B, 8C, and 8D depict CCR5⁺ cells within SUM159 activate DNA damage repair signaling pathways by gene expression.

FIGS. 9A, 9B, and 9C depict CCR5 increased DNA damage repair in SUM159 breast cancer cells showed by Phospho-γH2AX Western-blot.

FIGS. 10A, 10B, and 10C depict DNA repair reporter assays CCR5 induces activity conducted in SUM159 cells.

### DETAILED DESCRIPTION

The embodiments disclosed herein and the various features and advantages thereto are more fully explained with references to the non-limiting embodiments and examples that are described and set forth in the following descriptions of those examples. Descriptions of well-known components and techniques may be omitted to avoid obscuring the invention. The examples used herein are intended merely to facilitate an understanding of ways in which the embodiments may be practiced. Accordingly, the examples and embodiments set forth herein should not be construed as limiting the scope any of the appended claims.

As used herein, terms such as “a,” “an,” and “the” include singular and plural referents unless the context clearly demands otherwise.

As used in this document, terms “comprise,” “have,” “has,” and “include” and their conjugates, as used herein, mean “including but not limited to.” While various compositions, and methods are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

The phrase “pharmaceutically acceptable” or “therapeutically acceptable” refers to molecular entities and compositions that are physiologically tolerable and preferably do not typically produce an allergic or similar untoward reaction, such as gastric upset, dizziness and the like, when administered to a human. Preferably, as used herein, the term “pharmaceutically acceptable” means approved by a regulatory agency of the Federal or a State government or listed in the U.S. Pharmacopeia or other generally recognized pharmacopeia (e.g., Remington's Pharmaceutical Sciences, Mack Publishing Co. (A. R. Gennaro edit. 1985)) for use in animals, and more particularly in humans.

As used herein, the phrase “pharmaceutically acceptable salt(s),” includes, but is not limited to, salts of acidic or basic groups. Compounds that are basic in nature are capable of forming a wide variety of salts with various inorganic and organic acids. Acids that may be used to prepare pharmaceutically acceptable acid addition salts of such basic compounds are those that form non-toxic acid addition salts, i.e., salts containing pharmacologically acceptable anions including, but not limited to, sulfuric, thiosulfuric, citric, maleic, acetic, oxalic, hydrochloride, hydrobromide, hydroiodide, nitrate, sulfate, bisulfate, bisulfite, phosphate, acid phosphate, isonicotinate, borate, acetate, lactate, salicylate, citrate, acid citrate, tartrate, oleate, tannate, pantothenate, bitartrate, ascorbate, succinate, maleate, gentisinate, fumarate, gluconate, glucoronate, saccharate, formate, benzoate, glutamate, methanesulfonate, ethanesulfonate, benzenesulfonate, p-toluenesulfonate, bicarbonate, malonate, mesylate, esylate, napsydisylate, tosylate, besylate, orthophoshate, trifluoroacetate, and pamoate (i.e., 1,1′-methylene-bis-(2-hydroxy-3-naphthoate)) salts. Compounds that include an amino moiety may form pharmaceutically acceptable salts with various amino acids, in addition to the acids mentioned above. Compounds that are acidic in nature are capable of forming base salts with various pharmacologically acceptable cations. Examples of such salts include, but are not limited to, alkali metal or alkaline earth metal salts and, particularly, calcium, magnesium, ammonium, sodium, lithium, zinc, potassium, and iron salts. The present invention also includes quaternary ammonium salts of the compounds described herein, where the compounds have one or more tertiary amine moiety.

As used herein, the terms “treat,” “treated,” or “treating” mean both therapeutic treatment wherein the object is to slow down (lessen) an undesired physiological condition, disorder or disease, or obtain beneficial or desired clinical results. For purposes of the embodiments described herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment also includes prolonging survival as compared to expected survival if not receiving treatment. Thus, “treatment of cancer” or “treating cancer” means an activity that alleviates or ameliorates any of the primary phenomena or secondary symptoms associated with the cancer or any other condition described herein. In some embodiments, the cancer that is being treated is prostate or breast cancer.

As used herein, the term CCR5 refers to the C—C chemokine receptor type 5, which is a protein on the surface of cells that acts as a receptor for several chemokines, including CCL5, CCL7, and CCL8.

Breast tumor initiating cells conveying stem-like features contribute to therapeutic resistance and tumor metastases. Cancer stem cells are protected against chemotherapy and radiotherapy induced death, through mechanisms that protect genomic integrity via induction of DNA damage sensing and DNA repair machinery. The molecular mechanisms linking stem cells and DNA repair are poorly understood. Herein, the chemokine receptor CCR5, which is known to contribute to breast cancer progression and metastasis, was shown to promote stem cell-like properties and enhance DNA repair. Interestingly, the cytokine receptor CCR5 is normally expressed only on the surface of immune cells.

Reintroduction of CCR5 into CCR5-negative cells promoted breast tumor stem cell expansion, metastases, and the induction of DNA repair gene expression. CCR5 enhanced expression of DNA repair pathways of Fanconi anemia (FANCB, FANCE), basic excision repair (LIG3, DNA ATP-dependent ligase) and the HRR (XRCC2, X-Ray repair complementing defective repair in Chinese hamster cells 2). The finding that CCR5 augments DNA repair and cancer stem cell expansion has important implications for management of cancer therapy resistance.

Chemokine CCL5 and its receptor CCR5 play a significant role in cancer progression and metastasis. Metastasis is the primary cause of death in patients with breast cancer. No effective treatments currently exist that are directed specifically to the metastatic process. There is evidence that the CCR5 receptor is expressed in a subset of human breast cancers. Importantly, embodiments disclosed herein support that CCR5 inhibitors previously developed and FDA approved for treatment of HIV, can effectively block breast cancer metastasis in preclinical models, thereby providing for an effective treatment of said metastatic breast cancers. The repurposing of drugs for alternative use in cancer metastasis, drugs that were previously approved by the FDA, may provide a more rapid solution for this deadly disease. Indeed, CCR5 inhibition by the FDA approved CCR5 antagonists Maraviroc and Vicriviroc reduced in vivo metastasis in a basal-like breast cancer model.

Indeed, in studies described herein, CCR5 is sufficient to induce invasiveness of breast cancer cells into matrigel, and metastasis to the lungs of mice. Accordingly, two distinct CCR5 inhibitors, maraviroc and vicriviroc, blocked CCR5 signaling and thereby block migration, invasion and metastasis in mice. The CCR5 inhibitors were shown to block homing of breast cancer cells to the lungs. The dose of CCR5 inhibitor used in these mouse models was the same as the dose used in patients for HIV.

In embodiments described herein, oncogenic transformation of normal epithelial cells results in expression of CCR5. Therefore, and unexpectedly, CCR5 expressing transformed cells can be detected in the blood of humans, not simply at the site of the tumor, such as in the cancerous tissues. CCR5 expressing circulating tumor cells (CTC) can, therefore, be used to detect CCR5 expression. This expression can be used to detect cancer in a subject based upon the expression of CCR5 in a blood sample of the subject. The expression on CTCs can also be used to determine whether a subject will benefit from anti-CCR5 therapy, concomitant therapy with CCR5 inhibitor or agonist and a further therapeutic agent, and to monitor cancer treatment that targets CCR5 expression, among other embodiments.

Indeed, one problem is determining which cancer patients will benefit from CCR5-based therapy. No approved treatment for cancer currently involves CCR5 antagonists and no mechanism exists to determine CCR5 expression in cancer patients through routine sampling of blood. As described herein, CCR5-targeted therapy will be effective in cancer patients with tumors that express elevated levels of CCR5 and CCR5 ligands and elevated levels of circulating tumor cells (CTCs) and/or overt metastases. These elevated levels of CCR5 are found in all types of cancers. Results from studies disclosed herein show that the CCR5 is expressed upon oncogenic transformation of breast and prostate epithelial cells. (˜50% of breast cancer, 50% of prostate cancer). CCR5 is expressed on significant number of other therapy resistant human cancers including CCR5⁺ breast and prostate market about 250,000 patients yr. in the United States. Furthermore, CCR5 is shown to be positive in several other forms of metastatic cancers.

Accordingly, in some embodiments, CCR5 is utilized as a marker to define the chemotherapy and radiation therapy resistance of a tumor, such as breast and prostate. Combined with existing medicine, CCR5 blocking agents can be used to treat cancer patients to overcome this resistance. Therefore, CCR5 blocking agents can be used to treat cancer patients and further methods may be utilized to monitor treatment (surrogate measure of patient candidates and their therapy) based on the levels of CCR5 expression and/or CCR5 activity in CTC cells. For example, as CCR5 levels are decreased in CTCs or in the tumor sample itself, the tumor burden can be deteremined to be decreasing. Once the CCR5 levels are reduced to baseline, i.e. before the tumor was present or as compared to a normal control, the treatment with the CCR5 inhibitor or other type of cancer treatment can be stopped, reduced, or modified to a maintenance type therapy that is commonly used to treat cancers, such as breast and prostate.

In some embodiments, the CCR5 inhibitor can be utilized to determine efficacy of a CCR5 inhibitor in high-risk prostate cancer patients and other cancers that express CCR5.

The studies described herein indicate that activation of CCR5 in breast cancer cells contributes to resistance to several chemotherapeutic agents and promotes formation of breast tumor initiating cells (BTIC). The mechanism of resistance to current breast cancer chemotherapeutic agents is an urgent matter of broad importance to many patients with breast cancer, wherein new therapies would be available to treat certain cancers that are highly metastatic.

Accordingly, some embodiments are directed to methods for treating a metastatic cancer form that expresses CCR5 by treating said patient with an effective amount of a CCR5 inhibitor, sufficient to inhibit the level of CCR5 in the blood. Correspondingly, a further embodiment is directed to a method for determining patient candidates for treatment with CCR5 inhibitors by obtaining a blood sample from the patient and measuring the CCR5 expressed in circulating tumor cells. Patients that are positive for CCR5 expression in circulating tumor cells, therefore, can be treated with a CCR5 inhibitor. Methods for determining CCR expression on circulating tumor cells can be any method used to detect CCR5 expression, such as ELISA, microarray, antibody detection, and the like. In some embodiments, the mRNA of CCR5 can also be used as a proxy for CCR5 expression on the cell surface of the CTCs. Accordingly, in some embodiments, the mRNA from the CTCs is isolated and quantified. It can be measured by, for example, RT-PCR, microarray, and the like. CCR5 positive CTCs can also be detected using flow cytometry technology, such as FACS. Accordingly, in some embodiments, detecting the presence of CCR5 on circulating tumor cells comprises using flow cytometry to detect CCR5 on circulating tumor cells. In some embodiments, detecting the presence of CCR5 on circulating tumor cells comprises contacting an antibody that binds to CCR5 with a population of circulating tumor cells and detecting the bound antibody to determine the presence of CCR5 on circulating tumor cells. In some embodiments, a labeled ligand for CCR5 is used to detect the presence of CCR5 on the circulating tumor cells. The labeled ligan can be CCR5-L itself or another molecule that is capable of binding to CCR5. In some embodiments, detecting the presence of CCR5 on circulating tumor cells comprises performing an ELISA to detect the presence of CCR5 on circulating tumor cells. These are non-limiting embodiments and other methods and reagents can be used to detect the presence of CCR5 on circulating tumor cells.

CCR5 is highly upregulated in tumor cells and assists in repairing damaged DNA in these cells. Accordingly, the presence of CCR5 may be inversely correlated with the efficacy of cancer treatments that target cancerous cells by damaging their DNA, such as chemotherapy, radiation therapy, PARP inhibitors, etc. Because of the high levels of CCR5 in cancerous tissues, cancerous cells wherein CCR5 is present selectively repair damaged DNA much more efficiently than healthy cells, thus leading to reduced efficacy of treatment.

Further embodiments provide that a cancer treatment comprises a first step of inhibiting the CCR5 expression in the patient, followed by a treatment of standard course of radiation or chemotherapeutic agents after the CCR5 is inhibited. In some embodiments, the methods further comprises measuring CCR5 on CTCs to determine the efficacy of the treatment. In some embodiments, the subject is tested for CCR5 expression in CTCs found in the blood. Those patients that have CCR5 expression in the blood are determined to be susceptible to treatment with CCR5 inhibitors.

In the studies described herein, in some patients, circulating breast cancer tumor cells express CCR5. The presence of CCR5 on the patient's primary breast tumor can therefore be used as a companion diagnostic for therapeutic substratification and the patients CTC can be considered as a surrogate for monitoring treatment efficacy. As described herein, as the CCR5 levels decrease the treatment is determined to be effective.

Additional mechanistic studies to understand the contribution of CCR5 to metastasis and therapeutic resistance demonstrate that human breast cancers are heterogeneous for expression of CCR5 by FACS analysis and through cell separation of CCR5+ vs CCR5− cells from the same tumor we have shown that CCR5+ cells are enriched for cells with properties of breast cancer stem cells (increased mammosphere formation and epitope markers of cancer stem cells). Gene expression profiling of CCR5+ cells demonstrated activation of signaling pathways involved in DNA damage and repair. The dramatic enhancement of DNA repair signaling by CCR5 activation therefore suggests contribution to the resistance of a patient's tumor to chemotherapeutic agents. Accordingly, in some embodiments, the CCR5 inhibitors are administered with (concurrently or sequentially) with DNA damaging agents used to treat cancer. Examples of such DNA damaging agents include, but are not limited to, alkylating agents, intercalating agents, and polymerase inhibitors. Examples of such agents include, but are not limited to, busulfan, bendamustine, carboplatin, carmustine, chlorambucil, cisplatin, cyclophosphamide, dacarbazine, daunorubicin, decitabine, doxorubicin, epirubicin, etoposide, idarubicin, ifosfamide, irinotecan, lomustine, mechlorethamine, melphalan, mitomycin C, mitoxantrone, oxaliplatin, temozolomide, 5-fluorouracil, paclitaxel, and topotecan. Other agents, such as tamoxifen and related therapeutics may also cause DNA damage and can be combined with CCR5 inhibitors. In some embodiments, the DNA damaging agent is a combination of 5-fluorouracil, doxorubicin, and cyclophosphamide, which is commonly referred to as “FAC.” In some embodiments, the DNA damaging agent is a combination of paclitaxel, 5-fluorouracil, doxorubicin, and cyclophosphamide, which is commonly referred to as “TFAC.”

Other examples of DNA damaging agents include, but are not limited to, PARP inhibitors. Examples of PARP inhibitors include, but are not limited to, iniparib, talazoparib, niraparib, veliparib, olaparib, rucaparib, veliparib, CEP 9722, E7016 (Eisai), BGB-290, 3-aminobenzamide. As described herein, the CCR5 compounds can also be combined with radiation or other chemotherapeutics known to one of skill in the art. The DNA damaging agents can be used in any of the methods described herein. The DNA damaging agents, as described above, can be used at the same time or sequentially with the CCR5 inhibitors. In some embodiments, the CCR5 inhibitor is administered first, which is then followed by the DNA damaging agents, or vice versa. Without being bound to any particular theory, the use of the CCR5 inhibitor may make the cancer be more susceptible to the DNA damaging agent, prevent resistance to the DNA damaging agent, and the like. The treatments can be used in conjunction with the detection of CCR5 responsive cancers based upon the methods described herein.

Human breast cancers exhibit tumor heterogeneity. CCR5 expressing epithelial cells within a breast cancer promote tumor metastasis and characteristics of breast tumor initiating cells (BTIC). CCR5 expression, which was homogeneous in isogenic transformed human breast cancer cells, correlated with invasiveness and CCR5 inhibitors reduced breast cancer metastasis by inhibiting homing (1). It is therefore suggested that the CCR5+ cells within the breast tumor contribute to breast cancer metastasis. Therefore, in certain embodiments, treatment of patients can be determined based on CCR5+ cells within the breast tumor initiating cells, or found in circulating tumor cells, which are expressing CCR5. When CCR5 is found to be expressed in the CTCs, then a CCR5 inhibitor can be used to treat the cancer, such as breast and prostate.

In some embodiments, the CCR5 inhibitor or modulator is 4,4-difluoro-N-[(1S)-3-[(1R,5 S)-3-(3-methyl-5-propan-2-yl-1,2,4-tri-azol-4-yl)-8-azabicyclo[3.2.1]octan-8-yl]-1-phenylpropyl]cyclohexane-1-car-boxamide (“Maraviroc”), N-(1S)-3-3-(3-Isopropyl-5-methyl-4H-1,2,4-triazol-4-yl)-exo-8-azabi-cyclo[3.2.1]oct-8-yl-1-phenylpropylcyclobutanecarboxamide; N-(1S)-3-3-(3-Isopropyl-5-methyl-4H-1,2,4-triazol-4-yl)-exo-8-azabi-cyclo[3.2.1]oct-8-yl-1- phenylpropylcyclopentanecarboxamide; N-(1S)-3-3-3-(3-Isopropyl-5-methyl-4H-1,2,4-triazol-4-yl)-exo-8-azabicyclo[3.2.1]oct-8-yl-1-phenylpropyl-4,4,4-trifluorobutanamide; N-(1S)-3-3-(3-Isopropyl-5-methyl-4H-1,2,4-triazol-4-yl)-exo-8-azabi-cyclo[3.2.1]oct-8-yl-1-phenylpropyl-4,4-difluorocyclohexanecarboxamide; N-(1S)-3-3-(3-Isopropyl-5-methyl-4H-1,2,4-triazol-4-yl)-exo-8-azabi-cyclo[3.2.1] oct-8-yl-1 (3-fluorophenyl)propyl-4,4-difluorocyclohexanecarboxamide; and pharmaceutically acceptable salts or solvates thereof In some embodiments, the CCR5 compound is N-{3-[3-exo-(2-Methyl-1H-benzimidazol-1-yl)-8-azabicyclo[3.2.1]oct-8-yl]-1-phenylpropyl}cyclobutanecarboxamide; N-{(1S)-3-[3-exo-2-Methyl-1H-benzimidazol-1-yl)-8-azabicyclo[3.2.1]-oct-8-yl]-1-phenylpropyl}cyclobutanecarboxamide; N-{(1S)-3-[3-endo-(2-Methyl-1H-benzimidazol-1-yl)-8-azabicyclo[3.2.1]oct-8-yl]-1-phenylpropyl}cyclobutanecarboxamide; N-{(1S)-3-[3-exo-(2-Methyl-1H-benzimidazol-1-yl)-8-azabicyclo[3.2.1]oct-8-yl]-1-phenylpropyl}tetrahydro-2H-pyran-4-carboxamide; 1-Acetyl-N-{(1S)-3-[3-exo-(2-methyl-1H-benzimidazol-1-yl)-8-azabicy-clo[3.2.1]oct-8-yl]-1-phenylpropyl}3-azetidine carboxamide; 1-Hydroxy-N-{(1S)-3-[3-exo-2-methyl-1H-benzimidazol-1-yl)-8-azabicy-clo[3.2.1]oct-8-yl]-1-phenylpropyl}cyclopentanecarboxamide; 2-Methyl-N-{(1S)-3-[3-exo-(2-methyl-1H-benzimidazol-1-yl)-8-azabicy-clo[3.2.1]oct-8-yl]-1-phenylpropyl}cyclopropanecarboxamide; 2-Cyclopropyl-N-{1S)-3-[3-exo-(2-methyl-1H-benzimidazol-1-yl)-8-azabicyclo[3.2.1]oct-8-yl]-1-phenylpropyl}acetamide; N-{(1S)-3-[3-exo-(2-methyl-1H-benzimidazol-1-yl)-8-azabicyclo[3.2.1]oct-8-yl]-1-phenylpropyl}tetrahydro-3-furancarboxamide; 3,3,3-Trifluoro-N-{(1S)-3-[3-exo-(2-methyl-1H-benzimidazol-1-yl)-8-azabicyclo[3.2.1]oct-8-yl]-1-phenylpropyl}propanamide; N-{(1S)-3-[3-exo-(2-Methyl-1H-benzimidazol-1-yl)-8-azabicyclo[3.2.1]oct-8-yl]-1-phenylpropyl}tetrahydro-2-furancarboxamide; 1-(Acetylamino)-N-{(1S)-3-[3-exo-(2-methyl-1H-benzimidazol-1-yl)-8-azabicyclo[3.2.1]oct-8-yl]-1-phenylpropyl}cyclopentanecarboxamide; [0101] N-{(1S)-3-[3-exo-(2-Methyl-1H-benzimidazol-1-yl)-8-azabicyclo[3.2.1]oct-8-yl]-1-phenylpropyl}acetamide; 1-Methoxy-N-{(1S)-3-[3-exo-(2-methyl-1H-benzimidazol-1-yl)-8-azabicyclo[3.2.1]oct-8-yl]-1-phenylpropyl}cyclopentanecarboxamide; 1-Amino-N-{(1S)-3-[3-exo-(2-methyl-1H-benzimidazol-1-yl)-8-azabicyc-lo[3.2.1]oct-8-yl]-1-phenylpropyl}cyclopentanecarboxamide; 1-Methyl-N-{(1S)-3-[3-exo-(2-methyl-1H-benzimidazol-1-yl)-8-azabicy-clo[3.2.1]oct-8-yl]-1-phenylpropyl}-2-oxo-4-pyrrolidinecarboxamide; 1-Acetyl-N-{(1S)-3-[3-endo-(2-methyl-1H-benzimidazol-1-yl)-8-azabic-yclo[3.2.1]oct-8-yl]-1-phenylpropyl)3-azetidinecarboxamide; N-{(1S)-3-[3-endo-(2-Methyl-1H-benzimidazol-1-yl)-8-azabicyclo[3.2.1]oct-8-yl]-1-phenylpropyl}acetamide; N-{(1S)-3-[6-(2-Methyl-1H-benzimidazol-1-yl)-3-azabicyclo[3.1.0]hex-3-yl]-1-phenylpropyl}cyclobutanecarboxamide; 2-Cyclopropyl-N-{(1S)-3-[3-exo-(3-{4-[(methyl sulfonyl)amino]benzyl}-1,2,4-oxadiazol-5-yl)-8-azabicyclo[3.2.1]oct-8-yl]-1-phenylpropyl}acetamide; N-{(1S)-3-[7-exo-(2-Methyl-1H-benzimidazol-1-yl)-3-oxa-9-azabicyclo-[3.3.1]non-9-yl]-1-phenylpropyl}cyclobutanecarboxamide; [0110] 2-Cyclopropyl-N-{(1S)-3-[7-exo-(2-methyl-1H-benzimidazol-1-yl)-3-oxa-9-azabicyclo[3.3.1]non-9-yl]-1-phenylpropyl}acetamide; 3,3,3-Trifluoro-N-{(1S)-3-[7-exo-(2-methyl-1H-benzimidazol-1-yl)-3-oxa-9-azabicyclo[3.3.1]non-9-yl]-1-phenylpropyl}propanamide; N-{(1S)-3-[7-endo-(2-Methyl-1H-benzimidazol-1-yl)-3-oxa-9-azabicyclo[3.3.1]non-9-yl]-1-phenylpropyl}cyclobutanecarboxamide; 2-Cyclopropyl-N-{(1S)-3-[7-endo-(2-methyl-1H-benzimidazol-1-yl)-3-oxa-9-azabicyclo[3.3.1]non-9-yl]-phenylpropyl}acetamide; N-{(1S)-3-[7-exo-(2-Methyl-1H-benzimidazol-1-yl)-3-thia-9-azabicyclo[3.3.1]non-9-yl]-1-phenylpropyl}cyclobutanecarboxamide;2-Cyclopropyl-N-[(1S)-3-(3-endo-{[2-(4-fluorophenyl)acetyl]amino}-8-azabicyclo[3.2.1]oct-8-yl)-1-phenylpropyl]cyclobutanecarboxamide; N-[(1S)-3-(3-{[3-endo-(4-Fluorophenyl)propanoyl]amino}-8-azabicyclo-[3.2.1]oct-8-yl)-1-phenylpropyl]cyclobutanecarboxamide; N-[(1S)-3-(3-{[3-exo-(4-Fluorophenyl)propanoyl]amino}-8-azabicyclo[3.2.1]oct-8-yl)-1-phenylpropyl]cyclobutanecarboxamide; 2-Cyclopropyl-N-[(1S)-3-(3-exo-{[2-(4-fluorophenyl)acetyl]amino}-8-azabicyclo[3.2.1]oct-8-yl)-1-phenylpropyl]acetamide; N-{(1S)-3-[3-exo-(2-Methyl-1H-benzimidazol-1-yl)-8-azabi cyclo[3.2.1]oct-8-yl]-1-phenylpropyl)}1-propionyl-3-azetidinecarboxamide; N-{(1S)-3-[3-endo-(2-Methyl-1H-benzimidazol-1-yl)-8-azabicyclo[3.2.1]oct-8-yl]-1-phenylpropyl}tetrahydro-3-furancarboxamide; N-{(1S)-3-[3-endo-(2-Methyl-1H-benzimidazol-1-yl)-8-azabicyclo[3.2.1]oct-8-yl]-1-phenylpropyl}tetrahydro-2H-pyran4-carboxamide; N-{(1S)-3-[3-endo-(2-Methyl-1H-benzimidazol-1-yl)-8-azabicyclo[3.2.1]oct-8-yl]-1-phenylpropyl}tetrahydro-2-furancarboxamide; 1-Acetyl-N-{(1S)-3-[3-endo-(1H-benzimidazol-1-yl)-8-azabicyclo[3.2.1]oct-8-yl]-1-phenylpropyl}-3-azetidinecarboxamide; N-{(1S)-3-[3-endo-(1H-Benzimidazol-1-yl)-8-azabicyclo[3.2.1]oct-8-y-l]-1-phenylpropyl}-1-propionyl-3-azetidinecarboxamide; Methyl-3-[({(1S)-[3-exo-(2-methyl-1H-benzimidazol-1-yl)-8-azabicyclo[3.2.1]oct-8-yl]-1-phenylpropyl}amino)carbonyl]-1-azetidinecarboxylate; N-{(1S)-3-[3-endo-(2-Methyl-1H-benzimidazol-1-yl)-8-azabicyclo-[3.2-.1]oct-8-yl]-1-phenylpropyl}-1-propionyl-3-azetidinecarboxamide 1-Acetyl-N-{(1S)-3-[3-endo-(2-methyl-1H-benzimidazol-1-yl)-8-azabicyclo[3.2.1]oct-8-yl]-1-phenylpropyl}-2-azetidinecarboxamide; 2-[Acetyl (methyl)amino]-N-{(1S)-3-[3-endo-(2-methyl-1H-benzimidazol-1-yl)-8-azabicyclo[3.2.1]oct-8-yl]-1-phenylpropyl}acetamide; 3-[Acetyl(methyl)amino]-N-{(1S)-3-[3-endo-(2-methyl-1H-benzimidazol-1-yl)-8-azabicyclo[3.2.1]oct-8-yl]-1-phenylpropyl}propanamide; 2-Methoxy-N-{(1S)-3-[3-endo-(2-methyl-1H-benzimidazol-1-yl)-8-azabi-cyclo[3.2.1]oct-8-yl]-1-phenylpropyl}acetamide; 3-Methoxy-N-{(1S)-3-[3-endo-2-methyl-1H-benzimidazol-1-yl)-8-azabic-yclo[3.2.1]oct-8-yl]-1-phenylpropyl}propanamide; 1-Acetyl-N-{(1S)-3-[3-endo-(2-methyl-1H-benzimidazol-1-yl)-8-azabic-yclo[3.2.1]oct-8-yl]-1-phenylpropyl}-3-pyrrolidinecarboxamide; 1-Methyl-N-{(1S)-3-[3-endo-(2-methyl-1H-benzimidazol-1-yl)-8-azabic-yclo[3.2.1]oct-8-yl]-1-phenylpropyl}-2-oxo-4-pyrrolidinecarboxamide; 1-Acetyl-N-{(1S)-3-[3-exo-(2-ethyl-1H-benzimidazol-1-yl)-8-azabicyc-lo[3.2.1]oct-8-yl]-1-phenyylpropyl}-3-azetidinecarboxamide; N-{(1S)-3-[3-exo-(2-Ethyl-1H-benzimidazol-1-yl)-8-azabicyclo[3.2.1]-oct-8-yl]-1-phenylpropyl}-1-propionyl-3-azetidinecarboxamide; 1-Acetyl-N-((1S)-1-phenyl-3-{3-exo-[2-(trifluoromethyl)-1H-benzimid-azol-1-yl]-8-azabicyclo[3.2.1]oct-8-yl}propyl)-3-azetidinecarboxamide; N-((1S)-1-Phenyl-3-{3-exo-[2-(trifluoromethyl)-1H-benzimidazol-1-yl]-8-azabicyclo[3.2.1]oct-8-yl}propyl)-1-propionyl-3-azetidinecarboxamide; N-((1S)-1-Phenyl-3-{3-exo[2-(trifluoromethyl)-1H-benzimidazol-1-yl]-8-azabicyclo[3.2.1]oct-8-yl}propyl)acetamide; 2-[Acetyl(methyl)amino]-N-((1S)-1-phenyl-3-{3-exo-[2-(trifluorometh-yl)-1H-benzimidazol-1-yl]-8-azabicyclo[3.2.1]oct-8-yl}propyl)acetamide; 1-Acetyl-N-{(1S)-3-[3-exo-(1H-benzimidazol-1-yl)-8-azabicyclo[3.2.1]oct-8-yl]-1-phenylpropyl}-3-azetidinecarboxamide; N(1S)-3-[3-exo-(1H-Benzimidazol-1-yl)-8-azabicyclo[3.2.1]oct-8-yl]-1-phenylpropyl)-1-propionyl-3-azetidinecarboxamide; 1-acetyl-N(1S)-3-[3-exo-(5-fluoro-1H-benzimidazol-1-yl)-8-azabicyclo[3.2.1]oct-8-yl]-1-phenylpropyl)3-azetidinecarboxamide; N-{(1S)-3-[3-exo-(5-Fluoro-1H-benzimidazol-1-yl)-8-azabicyclo[3.2.1]oct-8-yl]-1-phenylpropyl}-1-propionyl-3-azetidinecarboxamide; 1-Acetyl-N-{(1S)-3-[3-exo-(5-fluoro-2-methyl-1H-benzimidazol-1-yl)-8-azabicyclo[3.2.1]oct-8-yl]-1-phenylpropyl}3-azetidinecarboxamide; N-{(1S)-3-[3-exo-(5-Fluoro-2-methyl-1H-benzimidazol-1-yl)-8-azabicy-clo[3.2.1]oct-8-yl]-1-phenylpropyl}-1-propionyl-3-azetidinecarboxamide; N-{(1S)-3-[3-exo-(2-methyl-1H-benzimidazol-1-yl)-8-azabicyclo[3.2.1]oct-8-yl]-1-phenylpropyl}-3-azetidinecarboxamide; 1-methyl-N-{(1S)-3-[3-exo-(2-methyl-1H-benzimidazol-1-yl)-8-azabicy-clo[3.2.1]oct-8-yl]-1-phenylpropyl}-3-azetidinecarboxamide; (2S)-1-acetyl-N-{(1S)-3-[3-exo-(2-methyl-1H-benzimidazol-1-yl)-8-azabicyclo[3.2.1]oct-8-yl]-1-phenylpropyl}-2-azetidinecarboxamide; (2R)-1-acetyl-N-{(1S)-3-[3-exo-(2-methyl-1H-benzimidazol-1-yl)-8-az-abicyclo[3.2.1]oct-8-yl]-1-phenylpropyl}-2-azetidinecarboxamide; 2-[acetyl(methyl)amino]-N-{(1S)-3-[3-exo-(2-methyl-1H-benzimidazol-1-yl)-8-azabicyclo[3.2.1]oct-8-yl]-1-phenylpropyl}acetamide; 3-[acetyl(methyl)amino]-N-{(1S)-3-[3-exo-(2-methyl-1H-benzimidazol-yl)-8-azabicyclo[3.2.1]oct-8-yl]-1-phenylpropyl}propanamide; 1-acetyl-N-{(1S)-3-[3-exo-(2-methyl-1H-benzimidazol-1-yl)-8-azabicyclo[3.2.1]oct-8-yl]-1-phenylpropyl}-3-pyrrolidinecarboxamide; N-{(1S)-3-[3-exo-(2-methyl-1H-benzimidazol-1-yl)-8-azabicyclo[3.2.1]oct-8-yl]-1-phenylpropyl}-1-(trifluoromethyl)cyclopropanecarboxamide; 2-methoxy-N-{(1S)-3-[3-exo-(2-methyl-1H-benzimidazol-1-yl)-8-azabicyclo[3.2.1]oct-8-yl]-1-phenylpropyl}acetamide; 3-methoxy-N-{(1S)-3-[3-exo-(2-methyl-1H-benzimidazol-1-yl)-8-azabicyclo[3.2.1]oct-8-yl]-1-phenylpropyl}propanamide; 1-Acetyl-N-{(1S)-3-[3-exo-(4-fluoro-2-methyl-1H-benzimidazol-1-yl)-8-azabicyclo[3.2.1]oct-8-yl]-1-phenylpropyl}-3-azetidinecarboxamide; N-{(1S)-3-[3-exo-(4-Fluoro-2-methyl-1H-benzimidazol-1-yl)-8-azabicy-clo[3.2.1]oct-8-yl]-1-phenylpropyl}-3-azetidinecarboxamide; 1-Methyl-N-{(1S)-3-[3-exo-(4-fluoro-2-methyl-1H-benzimidazol-1-yl)-8-azabicyclo[3.2.1]oct-8-yl]-1-phenylpropyl}-3-azetidinecarboxamide; N-{(1S)-3-[3-exo-(4-Fluoro-2-methyl-1H-benzimidazol-1-yl)-8-azabicy-clo[3.2.1]oct-8-yl]-1-phenylpropyl}-1-propionyl-3-azetidinecarboxamide; 2-Methoxy-N-{(1S)-3-[3-exo-(4-Fluoro-2-methyl-1H-benzimidazol-1-yl)-8-azabicyclo[3.2.1]oct-8-yl]-1-phenylpropyl}acetamide; N-{(1S)-3-[3-exo-(4-Fluoro-2-Methyl-1H-benzimidazol-1-yl)-8-azabicyclo[3.2.1]oct-8-yl]-1-phenylpropyl}acetamide; 3-Methoxy-N-{(1S)-3-[3-exo-(4-fluoro-2-methyl-1H-benzimidazol-1-yl)-8-yl]-1-phenylpropyl}propanamide; 2-[Acetyl(methyl)amino]-N-{(1S)-3-[3-exo-(4-fluoro-2-methyl-1H-benzimidazol-1-yl)-8-azabicyclo[3.2.1]oct-8-yl]-1-phenylpropyl}acetamide; 3-[Acetyl(methyl)amino]-N-{(1S)-3-[3-exo-(4-fluoro-2methyl-1H-benzi-midazol-1-yl)-8-azabicyclo[3.2.1]oct-8-yl]-1-phenylpropyl}propanamide; N-{(1S)-3-[3-exo-(4-fluoro-2-methyl-1H-benzimidazol-1-yl)-8-azabicyclo[3.2.1]oct-8-yl]-1-phenylpropyl}-3-methyl-3-oxetanecarboxamide; 3-Ethyl-N-{(1S)-3-[3-exo-(4-fluoro-2-methyl-1H-benzimidazol-1-yl)-8-azabicyclo[3.2.1]oct-8-yl]-1-phenylpropyl}-3-oxetanecarboxamide; N-{(1S)-3-[3-exo-(4-Fluoro-2-methyl-1H-benzimidazol-1-yl)-8-azabicyclo[3.2.1]oct-8-yl]-1-phenylpropyl}-3-oxetanecarboxamide; 3-Ethyl-N-{(1S)-3-[3-exo-(4-fluoro-1H-benzimidazol-1-yl)-8-azabicyclo[3.2.1]oct-8-yl]-1-phenylpropyl}-3-oxetanecarboxamide; N-{(1S)-3-[3-exo-(4-Fluoro-1H-benzimidazol-1-yl)-8-azabicyclo[3.2.1]oct-8-yl]-1-phenylpropyl}-3-methyl-3-oxetanecarboxamide; N-{(1S)-3-[3-exo-(4-Fluoro-1H-benzimidazol-1-yl)-8-azabicyclo[3.2.1]oct-8-yl]-1-phenylpropyl}-3-oxetanecarboxamide; N-{(1S)-3-[3-exo-(4-Fluoro-1H-benzimidazol-1-yl)-8-azabicyclo[3.2.1]oct-8-yl]-1-phenylpropyl}-3-azetidinecarboxamide; N-{(1S)-3-[3-exo-(4-Fluoro-1H-benzimidazol-1-yl)-8-azabicyclo[3.2.1]oct-8-yl]-1-phenylpropyl}-1-methyl-3-azetidinecarboxamide; 1-Acetyl-N-{(1S)-3-[3-exo-(4-fluoro-1H-benzimidazol-1-yl)-8-azabicyclo[3.2.1]oct-8-yl]-1-phenylpropyl}-3-azetidinecarboxamide; N-{(1S)-3-[3-exo-(4-Fluoro-1H-benzimidazol-1-yl)-8-azabicyclo[3.2.1]oct-8-yl]-1-phenylpropyl}-1-propionyl-3-azetidinecarboxamide; N-{(1S)-3-[3-exo-(4-Fluoro-1H-benzimidazol-1-yl)-8-azabicyclo[3.2.1]oct-8-yl]-1-phenylpropyl}-2-methoxyacetamide; N-{(1S)-3-[3-exo-(4-Fluoro-1H-benzimidazol-1-yl)-8-azabicyclo[3.2.1]oct-8-yl]-1-phenylpropyl}acetamide; N{-1S)-3-[3-exo-(4-fluoro-1H-benzimidazol-1-yl)-8-azabicyclo[3.2.1]-oct-8-yl]-1-phenylpropyl}-3-methoxypropanamide; 2-[Acetyl(methyl)amino]-N-{(1S)-3-[3-exo-(4-fluoro-1H-benzimidazol-1-yl)-8-azabicyclo[3.2.1]oct-8-yl]-1-phenylpropyl}acetamide; 3-[Acetyl (methyl)amino]-N-{(1S)-3-[3-exo-(4-fluoro-1H-benzimidazol-1-yl)-8-azabicyclo[3.2.1]oct-8-yl]-1-phenylpropyl}propanamide; and pharmaceutical acceptable salts thereof. Although the compounds are listed in a large group, the compounds can also be used or claimed individually. In some embodiments, the CCR5 compound is maraviroc (4,4-difluoro-N-{(1S)-3-[3-(3-isopropyl-5-methyl-4H-1,2,4-triazol-4-yl)-8-azabicyclo[3.2.1]oct-8-yl]-1-phenylpropyl}cyclohexanecarboxamide) or vicriviroc (5-({4-[(3S)-4-{2-methoxy-1-[4-(trifluoromethyl)phenyl]ethyl}-3-methylpiperazin-1-yl]-4-methylpiperidin-1-yl}carbonyl)-4,6-dimethylpyrimidine). CCR5 compounds are also described in U.S. Patent Publication No. 20130303512, which is hereby incorporated by reference in its entirety. A CCR5 compound can be an inhibitor or an agonist of CCR5.

In some embodiments, the CCR5 compound is administered with one or more DNA damaging agents, such as, but not limited to, those described herein. The combination therapy can be used to treat various types of cancer. For example, the combination can be used to treat, or manage a neoplasm or metastasis of the neoplasm. In some embodiments, the neoplasm is cancer. Exemplary cancers and related disorders that can be treated or managed in accordance with the exemplary embodiments described herein include, but are not limited to, leukemias, such as but not limited to, acute leukemia, acute lymphocytic leukemia, acute myelocytic leukemias, such as, myeloblastic, promyelocytic, myelomonocytic, monocytic, and erythroleukemia leukemias and myelodysplastic syndrome; chronic leukemias, such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to ductal carcinoma, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma; gallbladder cancers such as adenocarcinoma; cholangiocarcinomas such as but not limited to pappillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, prostatic intraepithelial neoplasia, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell carcinoma, adenocarcinoma, hypernephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas. In some embodiments, the cancer is breast or prostate cancer.

Administration of the compounds may be carried out using any method known in the art. For example, administration may be transdermal, parenteral, intravenous, intra-arterial, subcutaneous, intramuscular, intracranial, intraorbital, ophthalmic, intraventricular, intracapsular, intraspinal, intracisternal, intraperitoneal, intracerebroventricular, intrathecal, intranasal, aerosol, by suppositories, or oral administration. In some embodiments, a pharmaceutical composition can be for administration for injection, or for oral, pulmonary, nasal, transdermal, ocular administration.

For oral administration, the peptide or a therapeutically acceptable salt thereof can be formulated in unit dosage forms such as capsules or tablets. The tablets or capsules may be prepared by conventional means with pharmaceutically acceptable excipients, including binding agents, for example, pregelatinised maize starch, polyvinylpyrrolidone, or hydroxypropyl methylcellulose; fillers, for example, lactose, microcrystalline cellulose, or calcium hydrogen phosphate; lubricants, for example, magnesium stearate, talc, or silica; disintegrants, for example, potato starch or sodium starch glycolate; or wetting agents, for example, sodium lauryl sulphate. Tablets can be coated by methods well known in the art. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active compound.

For topical administration, the peptide can be formulated in a pharmaceutically acceptable vehicle containing 0.1 to 10 percent, preferably 0.5 to 5 percent, of the active compound(s). Such formulations can be in the form of a cream, lotion, sublingual tablet, aerosols and/or emulsions and can be included in a transdermal or buccal patch of the matrix or reservoir type as are conventional in the art for this purpose.

For parenteral administration, the compounds of the present invention are administered by either intravenous, subcutaneous, or intramuscular injection, in compositions with pharmaceutically acceptable vehicles or carriers. The compounds can be formulated for parenteral administration by injection, for example, by bolus injection or continuous infusion. Formulations for injection can be presented in unit dosage form, for example, in ampoules or in multi-dose containers, with an added preservative. The compositions can take such forms as suspensions, solutions, or emulsions in oily or aqueous vehicles, and can contain formulatory agents, for example, suspending, stabilizing, and/or dispersing agents. Alternatively, the active ingredient can be in powder form for constitution with a suitable vehicle, for example, sterile pyrogen-free water, before use.

For administration by injection, it is can be desired to use the compound(s) in solution in a sterile aqueous vehicle which may also contain other solutes such as buffers or preservatives as well as sufficient quantities of pharmaceutically acceptable salts or of glucose to make the solution isotonic. In some embodiments, the pharmaceutical compositions of the present invention may be formulated with a pharmaceutically acceptable carrier to provide sterile solutions or suspensions for injectable administration. In particular, injectables can be prepared in conventional forms, either as liquid solutions or suspensions, solid forms suitable for solution or suspensions in liquid prior to injection or as emulsions. Suitable excipients are, for example, water, saline, dextrose, mannitol, lactose, lecithin, albumin, sodium glutamate, cysteine hydrochloride, or the like. In addition, if desired, the injectable pharmaceutical compositions may contain minor amounts of nontoxic auxiliary substances, such as wetting agents, pH buffering agents, and the like. If desired, absorption enhancing preparations (e.g., liposomes) may be utilized. Suitable pharmaceutical carriers are described in “Remington's pharmaceutical Sciences” by E. W. Martin.

For administration by inhalation, the compounds may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound and a suitable powder base, for example, lactose or starch. For intranasal administration the compounds of the invention may be used, for example, as a liquid spray, as a powder or in the form of drops.

The combination therapies described herein are expected to have synergistic and additive therapeutic effects. Synergy is defined as the interaction of two or more agents so that their combined effect is greater than the sum of their individual effects.

In some embodiments, the goal is to determine and identify other tractable components also linked to CCR5 on the surface of CTC. For example, to follow CCR5 expressing CTC in the body (blood and other metastatic site). Indeed, the identification of, and development of targeted therapies to CTC using CCR5 and its associated proteins is highly valuable. Furthermore, based on the development of these therapies, patients with CCR5 positive CTC can be treated by targeting CCR5 associated proteins (expressing CCR5 associated proteins).

In some embodiments, CTCs are measured in the blood sample of a patient and stained for CCR5 using antibody and immunofluorescence. This presence of CCR5 makes a patient a candidate for treatment with CCR5 antagonists. CCR5 treatment efficacy monitored by downstream markers. As described above, CCR5 expression on CTCs can be determined by other methods.

In the studies and examples described herein, it is disclosed that CCR5 is expressed upon oncogenic transformation of breast and prostate epithelial cells. In about 50% of breast cancer patients, CCR5 and its ligand CCL5 are expressed in the breast tumor. CCR5 is also expressed in other cancers in addition to breast and prostate cancers and thus the market may be as large as other metastatic cancer. Accordingly, the present embodiments can be used to better treat cancers and to better pick effective treatments that should be administered. It was surprising and unexpected that CTCs could be detected in the blood of a subject and be used as a marker for identifying an effective treatment and to follow the effectiveness of a treatment.

In some embodiments, the compounds and compositions described herein can be administered to a patient in need thereof. As used herein, the phrase “in need thereof” means that the patient (animal or mammal) has been identified as having a need for the particular method or treatment. In some embodiments, the identification can be by any means of diagnosis, such as the detection of CCR5 on CTCs. In any of the methods and treatments described herein, the animal or mammal can be in need thereof. In some embodiments, the patient is a human.

Accordingly, treatment of certain cancer forms expressing CCR5 improved by the administration of a CCR5 inhibitor such as those described herein, including but not limited to, maraviroc or vicriviroc are provided. Each of which can be systemically administered to a patient. Appropriate doses can be empirically determined by a person of ordinary skill in the art.

In some embodiments, the CCR5 inhibitor is administered to a patient to first to reduce the activity of CCR5 before a concomitant treatment with a further therapy for cancer treatment is administered. Such treatment may comprise administration of said CCR5 inhibitor for several days to several weeks, or may be administered during a brief treatment phase over a few days, followed by a break of at least a few days, and then subsequent treatment. Such cycles can be repeated as appropriate until the cancer is abated. In some embodiments, the other cancer therapy, such as a DNA damaging agent is administered before the CCR5 inhibitor is administered. In some embodiments, the two agents are administered simultaneously.

In some embodiments, CCR5 is expressed selectively on cancer, but not normal cells. Without being bound to any particular theory, chemotherapy induces DNA damage to kill cancer cells, but these chemotherapy agents also cause DNA damage of normal cells. CCR5 induces DNA repair; however, because of the highly elevated levels of CCR5 in certain cancer cells, CCR5 expression reduces the efficacy of chemotherapy agents because the repair to DNA damage is heightened specifically at the tumor, not in the normal cells. CCR5 inhibitors block DNA repair induction. Therefore, CCR5 inhibitors can be used to enhance sensitivity to DNA damage inducing therapies. The CCR5 inhibitors can also be used with lower doses of DNA damaging inducing therapies.

The chemokine receptor CCR5 is normally expressed on the surface of immune cells and served as HIV co-receptor. In breast cancer cells, there is a CCR5+ population that possesses stem cell-like property and is more tumorigenic. Compared with CCR5− cells, the CCR5+ cells were enriched with DNA damage/repair gene signature. The role of CCR5 in DNA repair was proved by western-blot of phospho-γH2AX and a DNA repair reporter assay in breast cancer cells with overexpression of CCR5 upon doxorubicin and γ-radiation induced DNA damage. Inhibition of CCR5 by its antagonist Maraviroc and Vicriviroc abolished these effects. Based on these results, the expression of CCR5 maintains the stemness of breast cancer cells through increase the DNA repair activity. Based on the existing theory that the resistance of cancer cells to the chemotherapy and radiation therapy caused by the existence of cancer stem cells, the inhibition of CCR5 by CCR5 antagonists will sensitize the response of breast cancer cells to chemotherapy and radiation therapy, therefore increase the efficiency of DNA damage inducing chemotherapy treatment for CCR5 expressing cancers.

The problem is side effects of DNA inducing chemotherapy agents are due to DNA damage of normal cells. The studies show that CCR5+ cells are enriched for DNA repair genes and function. Accordingly, embodiments described herein reduce the DNA repair in cancer cells, to enhance the efficacy of DNA damage inducing chemotherapy agents to allow a reduction in chemotherapy dose, to reduce side effects of current treatments in CCR5+ cancers.

Accordingly, the presence of CCR5 in CTCs makes patient a candidate for treatment with CCR5 antagonists. In some embodiments CCR5 treatment efficacy can be monitored by CCR5 activity itself or downstream markers of CCR5 activity (CTCs CCR5 activity). In some embodiments, CCR5 inhibitors are combined with traditional DNA damage inducing agents, which allows a reduction in dose of chemotherapy. The CCR5 inhibitors can be formulated and/or administered as a single dosage unit or as separate dosage units with the DNA damage based therapies

Accordingly, based upon the foregoing and below, various methods are also provided herein.

For example, in some embodiments, methods of treating a subject with cancer are provided. In some embodiments, the methods comprising detecting the presence of CCR5 on circulating tumor cells in a subject's sample; and administering to the subject with CCR5 on the circulating tumors cells a CCR5 inhibitor to treat the cancer. As described herein, the CCR5 on the circulating tumor cells can be detected by various methods. The CCR5 inhibitor can be, for example, the one of the many described herein. In some embodiments, the method further comprises administering a DNA damaging agent such as, but not limited to, those described herein. In some embodiments, the circulating tumor cell is a breast cancer circulating tumor cell. In some embodiments, the circulating tumor cell is a prostate cancer circulating tumor cell. In some embodiments, the CCR5 inhibitor is 4,4-difluoro-N-[(1S)-3-[(1R,5S)-3-(3-methyl-5-propan-2-yl-1,2,4-triazol-4-yl)-8-azabicyclo[3.2.1]octan-8-yl]-1-phenylpropyl]cyclohexane-1-carboxamide; N-(1S)-3-3-(3-Isopropyl-5-methyl-4H-1,2,4-triazol-4-yl)-exo-8-azabicyclo[3.2.1]oct-8-yl-1-phenylpropylcyclobutanecarboxamide; N-(1S)-3-3-(3-Isopropyl-5-methyl-4H-1,2,4-triazol-4-yl)-exo-8-azabicyclo[3.2.1]oct-8-yl-1-phenylpropylcyclopentanecarboxamide; N-(1S)-3-3-(3-Isopropyl-5-methyl-4H-1,2,4-triazol-4-yl)-exo-8-azabicyclo[3.2.1]oct-8-yl-1-phenylpropyl-4,4,4-trifluorobutanamide; N-(1S)-3-3-(3-Isopropyl-5-methyl-4H-1,2,4-triazol-4-yl)-exo-8-azabicyclo[3.2.1]oct-8-yl-1-phenyl propyl-4,4-difluorocyclohexanecarboxamide; N-(1S)-3-3-(3-Isopropyl-5-methyl-4H-1,2,4-triazol-4-yl)-exo-8-azabicyclo[3.2.1]oct-8-yl-1-(3-fluorophenyl)propyl-4,4-difluorocyclohexanecarboxamide; and pharmaceutically acceptable salts or solvates thereof. In some embodiments, the CCR5 inhibitor is maraviroc or vicriviroc.

In some embodiments, the methods further comprise obtaining the subject's sample. In some embodiments, the sample is a blood sample. In some embodiments, the sample is a urine sample. In some embodiments, the sample is fluid extract from the lymph system or from a lymph node. In some embodiments, the sample is serum free. In some embodiments, the sample is free of plasma. In some embodiments, the sample consists essentially of circulating tumor cells. In some embodiments, the sample has been enriched for circulating tumor cells. Enrichment can be performed by isolating tumor cells based upon tumor specific antigens, such as CD44, HER2, MUC-1, Carcinoembryonic antigen (CEA), Tn, TF, and sialyl-Tn (STn) antigens, CD107b, CD51, and CD61 and the like. Enrichment can be performed by any method, such as flow cytometry or other types of selection methods known to one of skill in the art. The enriched sample can then be analyzed for the presence of CCR5 on the circulating tumor cells.

Also provided, in some embodiments, are methods of identifying and treating a cancer in a subject as susceptible to a CCR5 inhibitor. In some embodiments, the method comprises obtaining a sample from the subject, detecting the presence of CCR5 on circulating tumor cells in the subject's sample; and identifying the cancer as susceptible to a CCR5 inhibitor when CCR5 is found to be present on the circulating tumor cells; and administering to the identified subject a CCR5 inhibitor to treat the cancer. In some embodiments, the method further comprises administering a DNA damaging agent. The cancer can be any type of cancer as described herein including, but not limited to, breast cancer or prostate cancer.

In some embodiments, methods of treating a subject with cancer are provided. In some embodiments, the methods comprise detecting the presence of CCR5 on circulating tumor cells in a subject's sample; and administering to the subject with CCR5 on the circulating tumors cells a CCR5 inhibitor and a DNA damaging agent to treat the cancer. In some embodiments, the CCR5 inhibitor is maraviroc or vicriviroc or other CCR5 inhibitor or compound described herein. In some embodiments, the DNA damaging agent is an alkylating agent, intercalating agents, or a polymerase inhibitor. In some embodiments, the DNA damaging agent is busulfan, bendamustine, carboplatin, carmustine, chlorambucil, cisplatin, cyclophosphamide, dacarbazine, daunorubicin, decitabine, doxorubicin, epirubicin, etoposide, idarubicin, ifosfamide, irinotecan, lomustine, mechlorethamine, melphalan, mitomycin C, mitoxantrone, oxaliplatin, temozolomide, 5-fluorouracil, paclitaxel, and topotecan. In some embodiments, the DNA damaging agent is a PARP inhibitor. In some embodiments, the DNA damaging agent is a combination of 5-fluorouracil, doxorubicin, and cyclophosphamide, which is commonly referred to as “FAC.” In some embodiments, the DNA damaging agent is a combination of paclitaxel, 5-fluorouracil, doxorubicin, and cyclophosphamide, which is commonly referred to as “TFAC.”

In some embodiments, methods of detecting cancer in a subject are provided. In some embodiments, the methods comprise detecting CCR5 expression on circulating tumor cells in a blood sample obtained from the subject, wherein the cancer is detected when CCR5 expression is detected on the circulating tumor cells in the blood sample obtained from the subject. In some embodiments, the cancer that is detected is breast or prostate cancer. In some embodiments, the methods further comprise administering a CCR5 inhibitor to the subject to treat the detected cancer. In some embodiments, the methods further comprise administering a DNA damaging agent to the subject to treat the detected cancer.

In some embodiments, methods of treating a chemotherapeutic resistant or radiation resistant tumor in a subject are provided. In some embodiments, the methods comprise administering to the subject a CCR5 inhibitor alone or in conjunction with a DNA damaging agent. In some embodiments, the method comprises identifying the subject with the chemotherapeutic resistant or radiation resistant tumor as susceptible to a CCR5 inhibitor, wherein the identifying comprises obtaining a sample from the subject, detecting the presence of CCR5 on circulating tumor cells in the subject's sample, and identifying the chemotherapeutic resistant or radiation resistant tumor as susceptible to a CCR5 inhibitor when CCR5 is found to be present on the circulating tumor cells. A resistant tumor is one that no longer response to either the chemotherapy or radiation that was used to initially treat the cancer. The cancer's resistance can be overcome by combining the treatment that was initially used with a CCR5 inhibitor, such as those described herein. For example, a patient with breast cancer is treated with doxorubicin. The breast cancer initially responds to the treatment, but then becomes resistant. The resistant tumor is then treated with a combination of the doxorubicin and a CCR5 inhibitor. The resistance is then overcome and the breast cancer responds to the combination treatment. This is only one example of such resistance that can be overcome by combining cancer treatments with CCR5 inhibitors. The resistance to FAC and TFAC may also be overcome with the addition of CCR5 inhibitors.

In some embodiments, methods of monitoring the effectiveness of a treatment on a CCR5 positive cancer are provided. In some embodiments, the method comprises administering a cancer treatment to a subject with a CCR5 positive cancer; and detecting CCR5 expression on circulating tumor cells in a sample obtained from the treated subject, wherein a decrease in CCR5 expression on the circulating tumor cells as compared to a sample from the subject before treatment indicates that the treatment is effective on the CCR5 positive cancer. In some embodiments, the treatment is a CCR5 inhibitor. In some embodiments, the treatment is a DNA damaging agent. In some embodiments, the treatment is a combination of a CCR5 inhibitor and a DNA damaging agent.

In order that the embodiments disclosed herein may be more efficiently understood, examples are provided below. It should be understood that these examples are for illustrative purposes only and are not to be construed as limiting.

### EXAMPLES

Several studies were performed to evaluate the expression of CCR5 in IBC cell lines and to demonstrate an antimetastatic effect in IBC preclinical models IBC cell lines SUM149, SUM190, and FC-IBC-02 derived from pleural effusion fluid of an IBC patient were examined for the CCR5 expression by immunofluorescence staining.

The effects of CCR5 antagonists (Maraviroc and Vicriviroc) on cell migration and invasion in vitro were examined using matrigel-coated Boyden chamber assay in FC-IBC-02 cells, which are triple-negative, basal-like, cancer stem cell phenotype, and rapidly developed primary tumors and metastasis in vivo. Cells were treated by Maraviroc and Vicriviroc at 100 μM and support the conclusion that CCR5 is highly expressed in basal-like human IBC cells.

Indeed, CCR5 antagonists demonstrated inhibition of migration and invasion. These results suggest an antimetastatic role for CCR5-inhibitors in IBC that can be effectively treated by administering to a patient an effective amount of CCR5 antagonist or inhibitors.

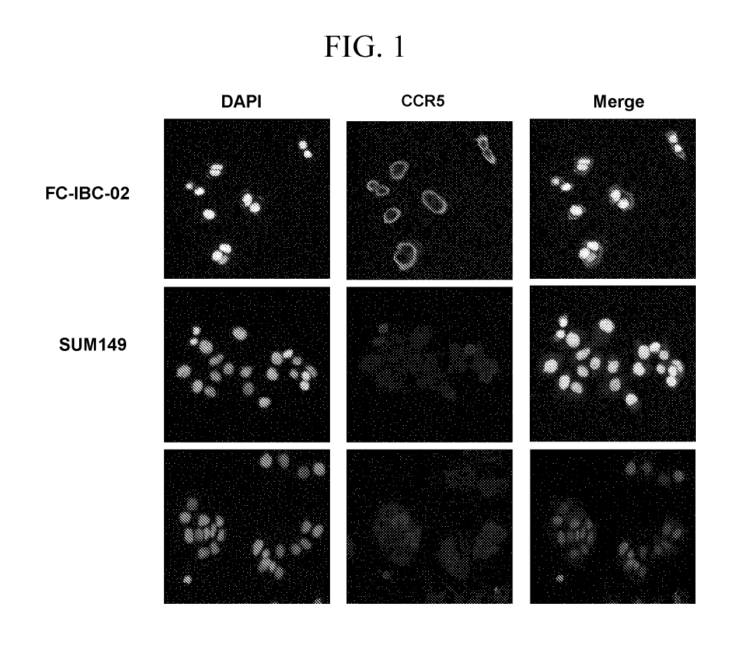

FIG. 1 depicts that CCR5 is expressed at higher levels in basal-like human inflammatory breast cancer (IBC) cell line FB-IBC-02 cells. CCR5 was expressed at low positive component comprising only about 5-7% of the total cell population in SUM 149 cells. CCR5 expression was not detected in SUM190 cells. CCR5 was expressed at significantly higher levels with a higher percentage of positive population (50-60%) in FC-IBC-02 cells compared with SUM149 cells.

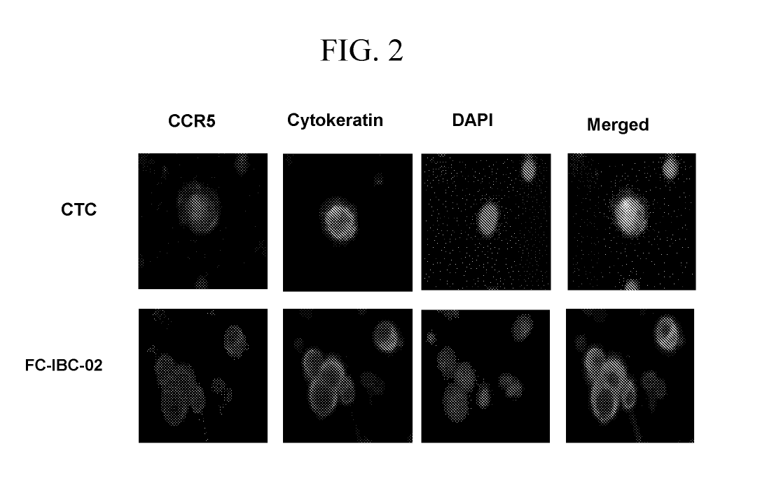

As depicted in FIG. 2, CCR5 expression was detected in circulating tumor cell (CTC) of IBC patient peripheral blood sample. Immunofluorescence staining shows that both Cytokeratin and CCR5 were expressed in FC-IBC-02 and CTC cells.

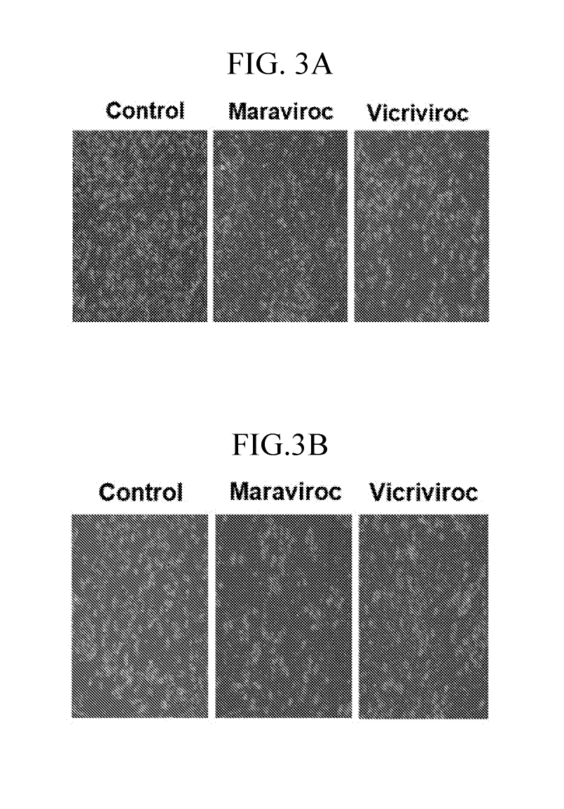

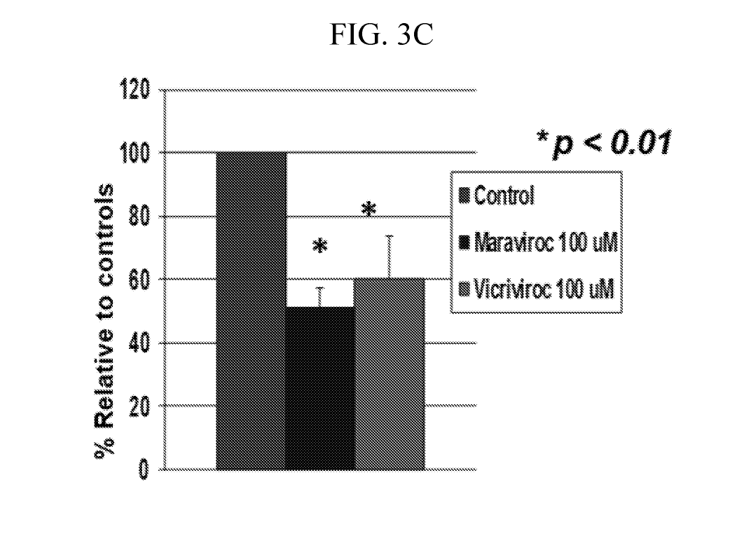

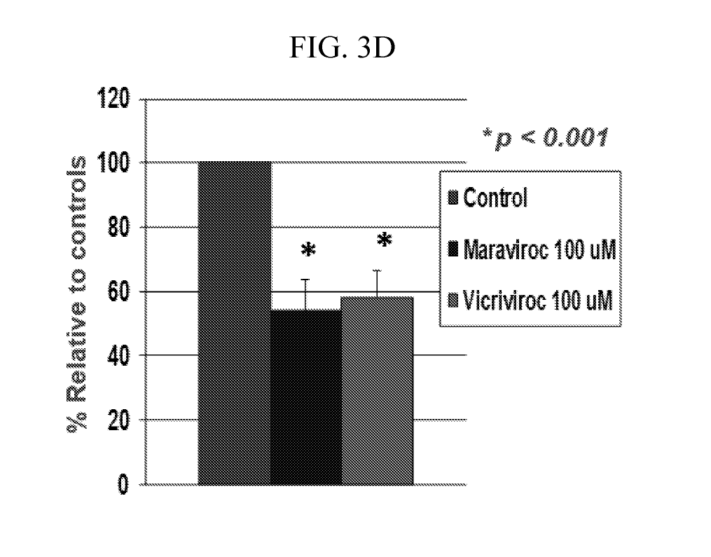

In FIG. 3, CCR5 antagonists Maraviroc and Vicriviroc suppress the migration and invasion of FC-IBC-02 cells. FIGS. 3A and 3B depict control and CCR5 antagonists treated cells at equal number were seeded onto the noncoated membrane (migration) and matrigel-coated membrane (invasion). After incubating 48 hrs. the cells crossed the membrane were stained with 0.05% crystal violet. FIGS. 3C and 3D, quantitation of 3A and 3B compared to controls was indicated. Maraviroc and Vicriviroc demonstrated effective agents in controlling migrations and invasion. *P value compared to control.

Accordingly, FIGS. 1-3 provide evidence that CCR5 is highly expressed in basal-like human IBC cells and that CCR5 antagonists demonstrate inhibition of migration and invasion. These results suggest that CCR5 inhibitors are suitable in an antimetastatic role for CCR5 in IBC. Furthermore, because of the demonstrated effects, in IBC, other cancers that express CCR5 can also be treated with the same or similar CCR5 antagonists.

In previous studies, MDA-MB231 cells were shown to express CCR5 and their metastasis to lung is blocked by CCR5 inhibitor (1, 7). The SUM159 breast cancer cell lines have been stably transduced with the LUC2 gene to allow detection of small numbers of cells in the metastatic sites and in the circulation. FACS analysis of several different human breast cancers demonstrated that the abundance of CCR5 is heterogeneous in human breast cancer epithelial cells.

Based on initial data and studies, CCR5 expression on human breast cancer cells induces gene expression and thereby signaling pathways that promote DNA repair and thereby resistance to chemotherapeutic resistance. Therefore, selection of CCR5+ vs CCR5− cells from within human breast cancer by FACS analysis demonstrated enrichment for gene signaling of DNA repair pathways and enhanced formation of BTIC using several complementary assays.

Therefore, therapeutic resistance to DNA damaging agents is associated with activation of DNA repair pathways. DNA repair pathways are activated in BTIC. Several chemotherapeutic agents function through inducing DNA damage including PARP inhibitors. (There may be two classes of PARP inhibitors, catalytic inhibitors that act mainly to inhibit PARP enzyme activity and do not trap PARP proteins on DNA, and dual inhibitors that both block PARP enzyme activity and act as PARP poison). Chemotherapy resistance is associated with increased gene expression of cytokine signaling.

Numerous cancer patient treatments relapse due to the drug resistance and radiation resistance in part because of increased DNA repair in either the tumor or the cancer stem cells of the tumor. CCR5 increases cancer stem cell number and increase DNA repair. Thus regulation of CCR5 to eliminate or modify the levels of CCR5 provide that a CCR5 inhibitor will reduce the DNA repair and cancer stem cells and thereby improve therapeutic response of current agents. Without being bound to any particular theory, this is the basis for adding CCR5 inhibitor to a current therapy based on DNA based induction.

Therefore, a combined therapy increases the efficacy of the primary treatment and allows for several therapeutic options, including maintenance doses and increasing the efficacy and kill rate of cancerous cells, or reducing the dose of the primary therapy in view of the increased efficacy through the use of the CCR5 inhibitor for patients that are CCR5 positive.

The studies described herein provide extensive information about the cohort of 2,000 breast cancer specimens including molecular subtyping into Luminal A, Luminal B, Mixed luminal and basal (mixed ER+/CK5+), Her2-positive, Basal, and Stem-like (claudin-low) breast cancer subclasses. Accordingly, the studies describe the distribution of CCR5 and ligands within the various predefined subtypes using qIF.

Indeed, a subgroup of breast cancer display an autocrine loop of self-stimulation of CCR5 by co-expression of one or more of the CCR5 ligands. This type of breast cancer is expected to be autonomously under CCR5 ligand control both in the primary tumor environment and in metastatic sites. CCR5-positive cancer cells that rely on paracrine CCR5 ligands from neighboring cells in the tumor microenvironment may still respond to CCR5 targeting but require seeding in metastatic sites that express CCR5 ligands for continued activation of CCR5. A third group of cancers that do not express CCR5 in the carcinoma cells are the least likely candidates for response to CCR5 inhibitors. Therefore, methods of identifying tumors with increased likelihood of responding to CCR5-targeted therapies can be used to determine potential responders to CCR5 treatment, wherein a subsequent step comprises treating said patients with a CCR5 inhibitor, and then monitoring said treatment, by measuring CCR5 response within CTC cells in the blood.

Data from the studies described herein supports that elevated protein levels of CCR5 and CCR5 ligands in breast cancer cells are associated with increased risk of aggressive disease, including metastases, early disease recurrence, poor overall survival, and elevated circulating tumor cell (CTC) counts at baseline, CCR5-positive CTCs. Therefore, based on mRNA levels of CCR5 and a CCR5-related gene signature support the hypothesis that elevated CCR5 expression and activity is associated with metastasis and poor clinical outcome in basal, Her2+ and even some ER+ tumors breast cancer patients.

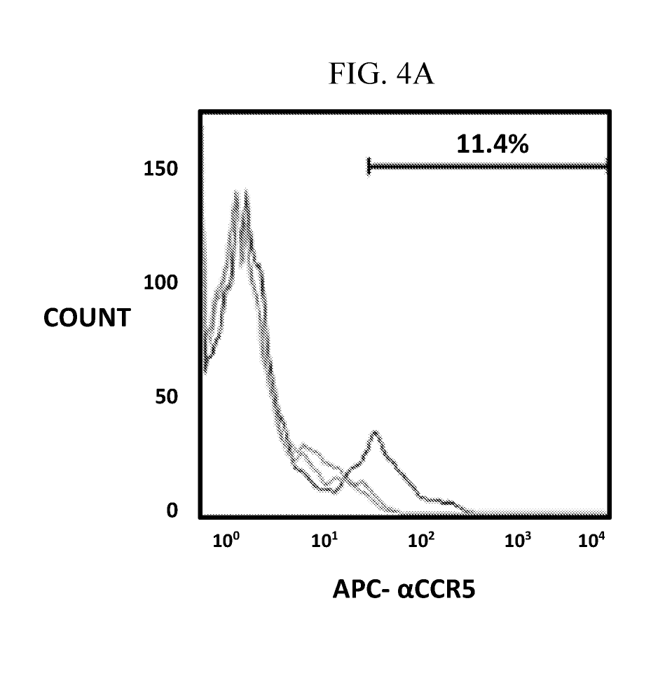

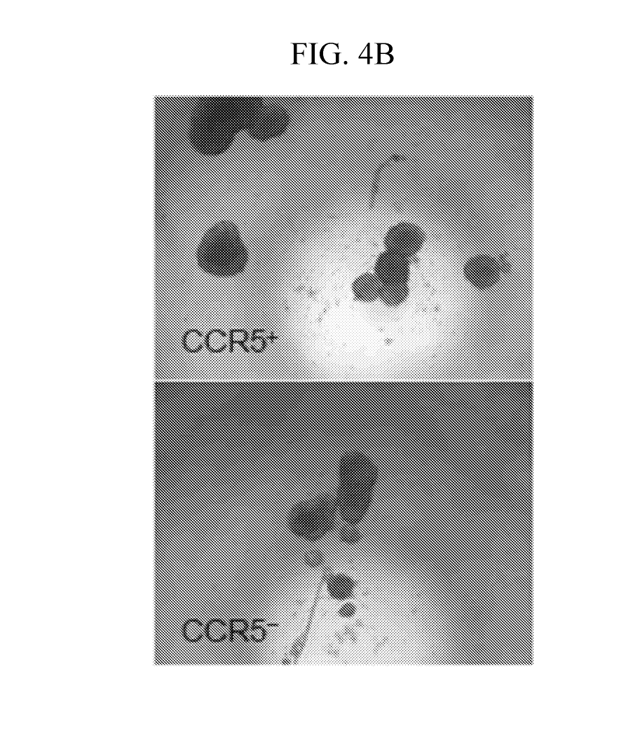

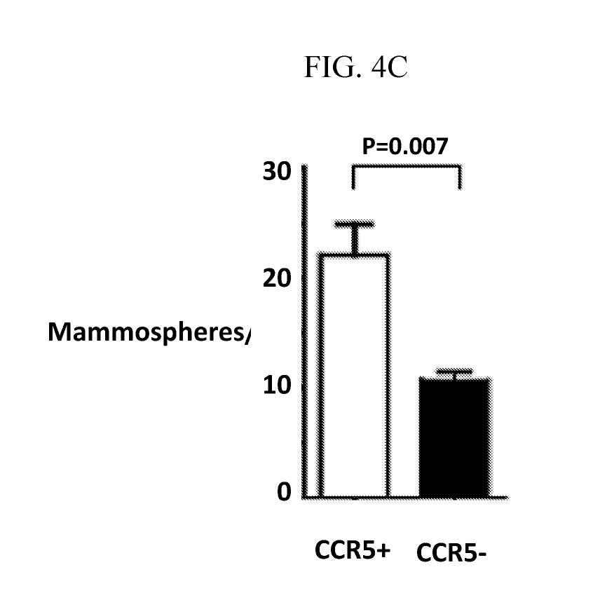

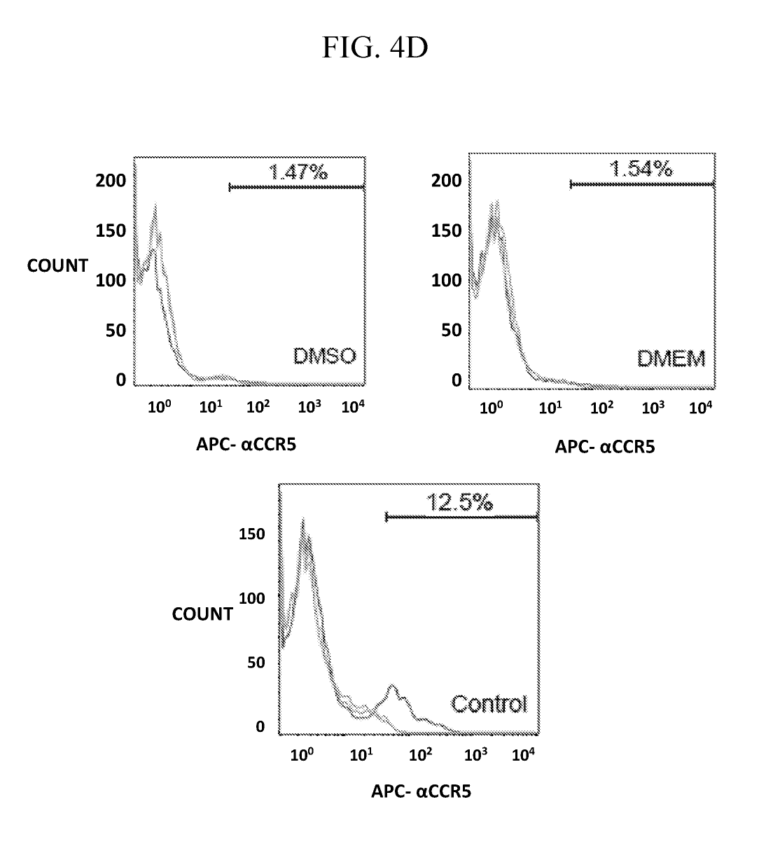

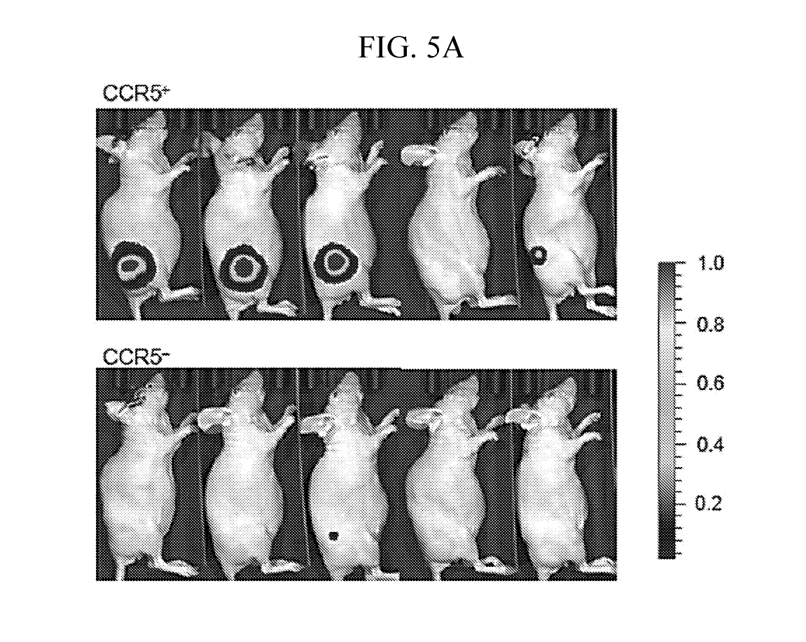

In order to determine the role of CCR5 in tumor initiating cells, the basal breast cancer subtype SUM159 cells were assessed. Consistent with the known tumor heterogeneity, fluorescence activated cell sorting (FACS) identified a subset of CCR5 positive cells within SUM159 (FIGS. 4A, 4B). The CCR5+ and CCR5− cells were selected using FACS. A surrogate assay of stem cell properties (mammosphere formation) was conducted (32, 36). The number of mammospheres represent propensity towards progenitor cell expansion. The relative proportion of mammospheres were increased 2.5 fold in the CCR5+/+ vs. CCR5− cells (FIGS. 4B, 4C) (P=0.007).

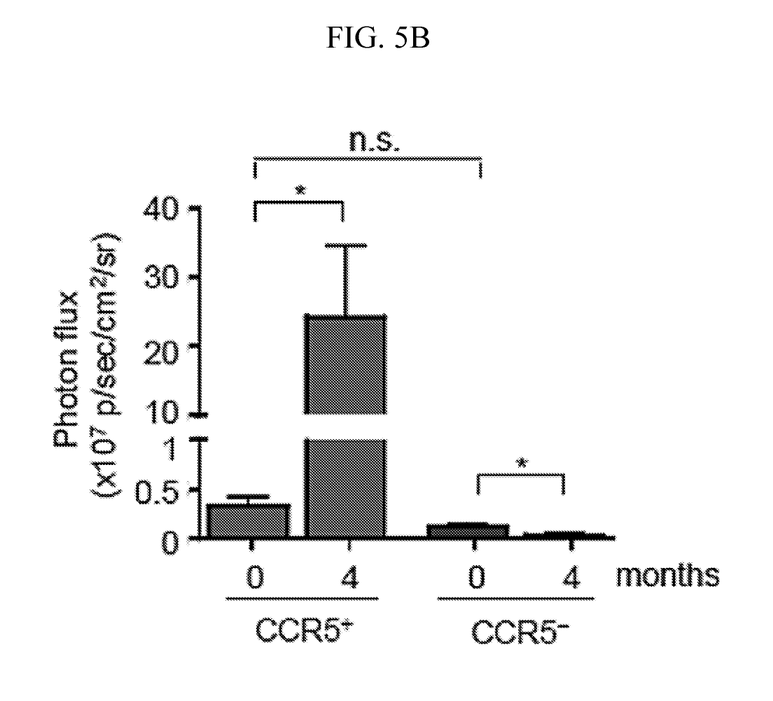

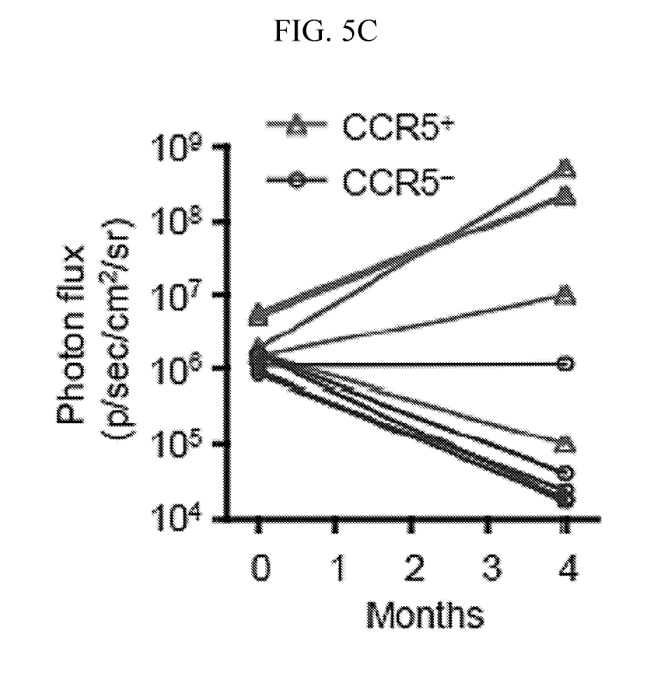

CCR5+ cells convey metastatic propensity. In order to characterize the metastatic properties of CCR5+ vs. CCR5− cells within the SUM159 breast cancer cells, FACS analysis was used to define a CCR5+ vs. CCR5− population. An equal number of cells were introduced into nude mice and the propensity towards metastatic lung formation was determined using bioluminescence which showed as photon flux of the cell lines. The CCR5+ subpopulation of SUM159 cells developed substantial tumors, increasing >40 fold over 4 months. In contrast, the CCR5− population declined in size between the same time period (FIGS. 5B, C). These studies are consistent with a role for CCR5 in tumor cell survival and growth.

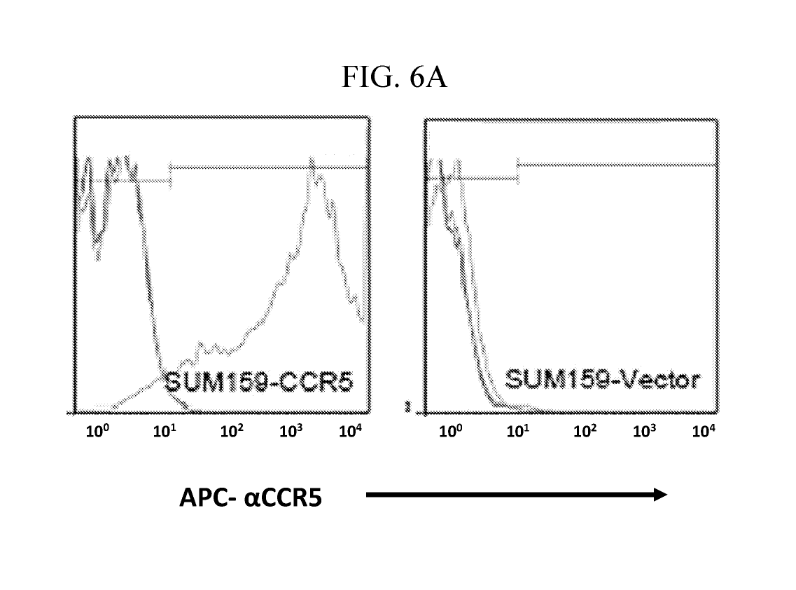

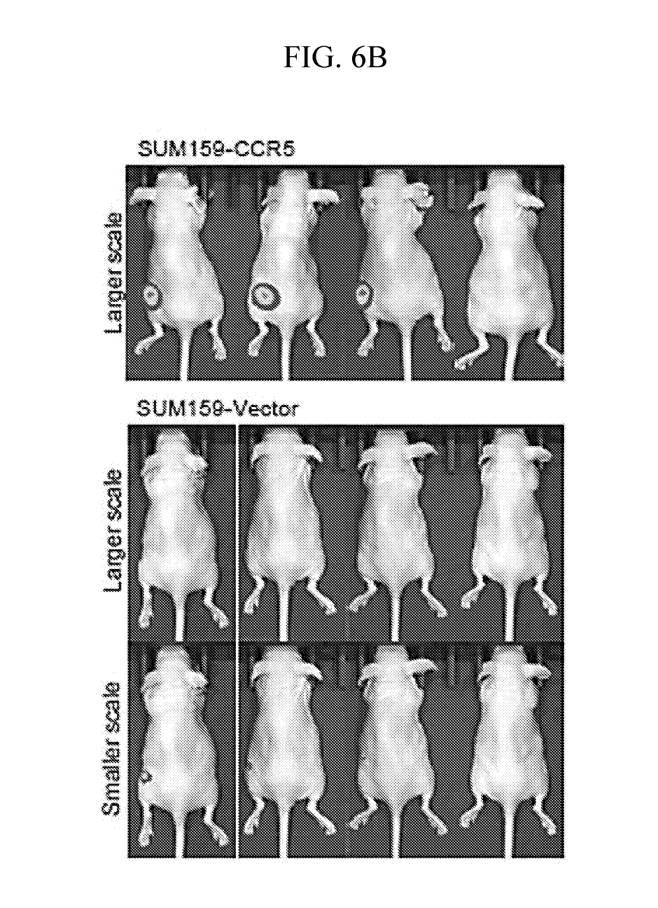

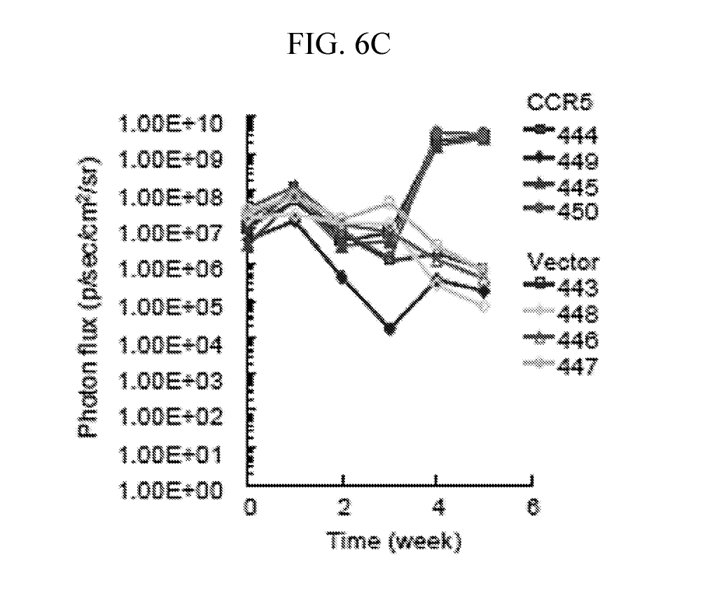

In order to examine further whether CCR5 was sufficient for enhancement of tumor growth, the SUM159− cells were stably transduced with a lentivirus based plasmidencoding CCR5. A comparison was made to empty vector control SUM159 stable lines. Tumor growth was examined over 6 weeks. The mean size of tumor volume was expressed using photon flux and expressed on a log scale. The size of tumors was enhanced 1×10⁴ times (FIG. 6B). Together, these studies demonstrate either endogenous CCR5 or reintroduction of CCR5 is sufficient for the induction of basal breast cancer cellular tumor growth in vivo.

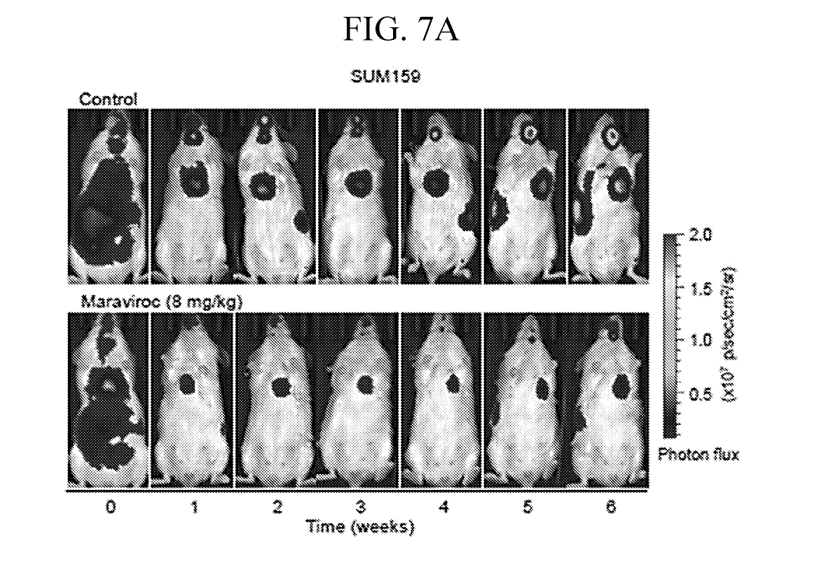

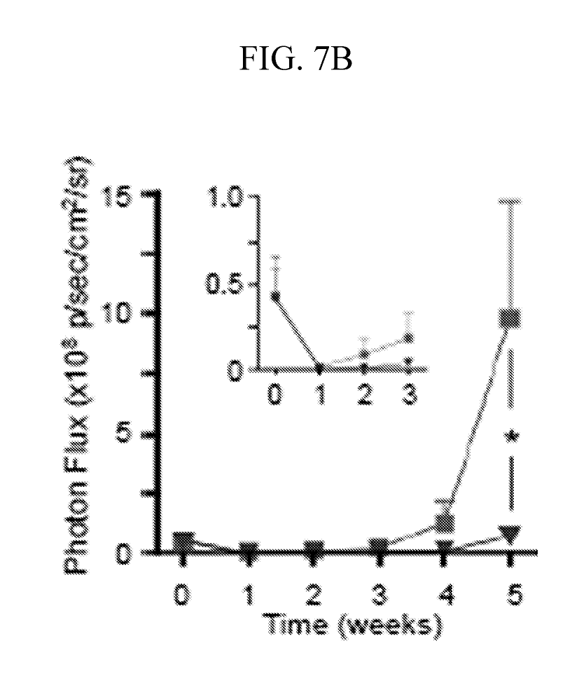

The CCR5 antagonist, Maraviroc, was previously approved by the US Food and Drug Administration (FDA) for the use in treatment-nave adult cells with CCR5-trophic HIV. In order to determine the role endogenous CCR5 within SUM159 cells in lung metastasis, cells were introduced into SCID immune deficient mice, and tumor volume characterized using the IVIS system. Lung metastases quantified using photon flux demonstrated a 15-fold increase in breast cancer lung metastases in the control group compared to the Maraviroc treated group (FIG. 7A, B). The percentage of mice with detectable lung metastases was significantly reduced with the CCR5 inhibitor Maraviroc administered orally. (p<0.0001).

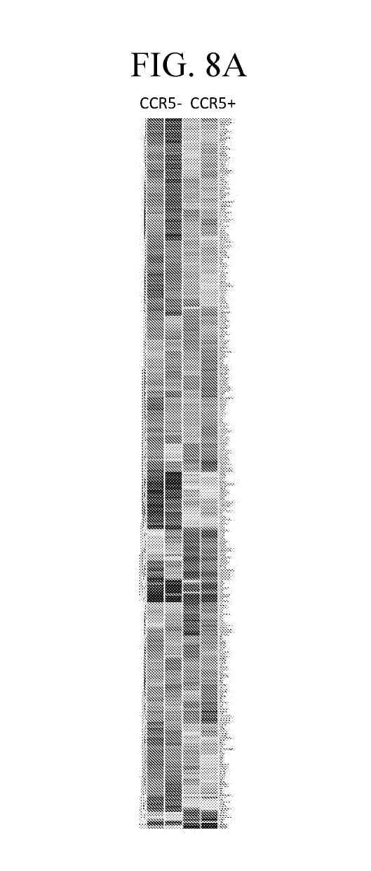

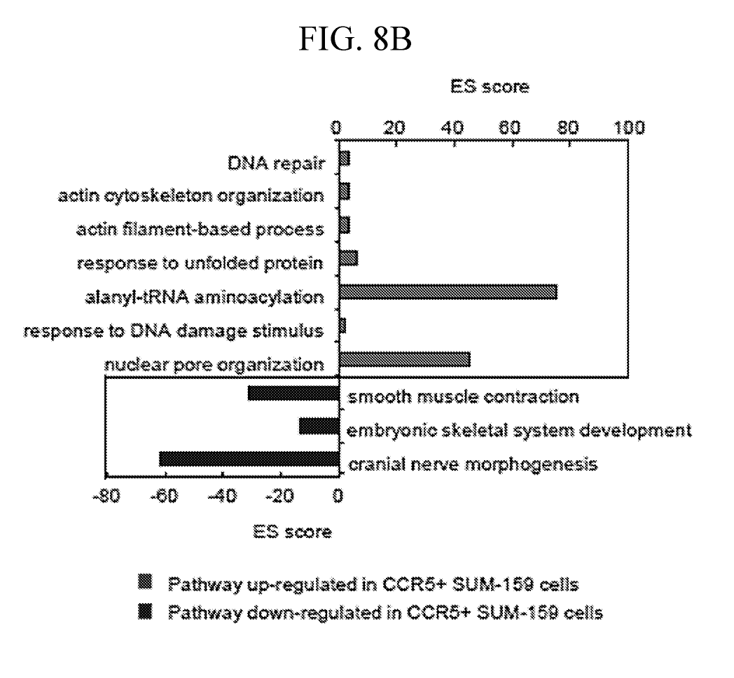

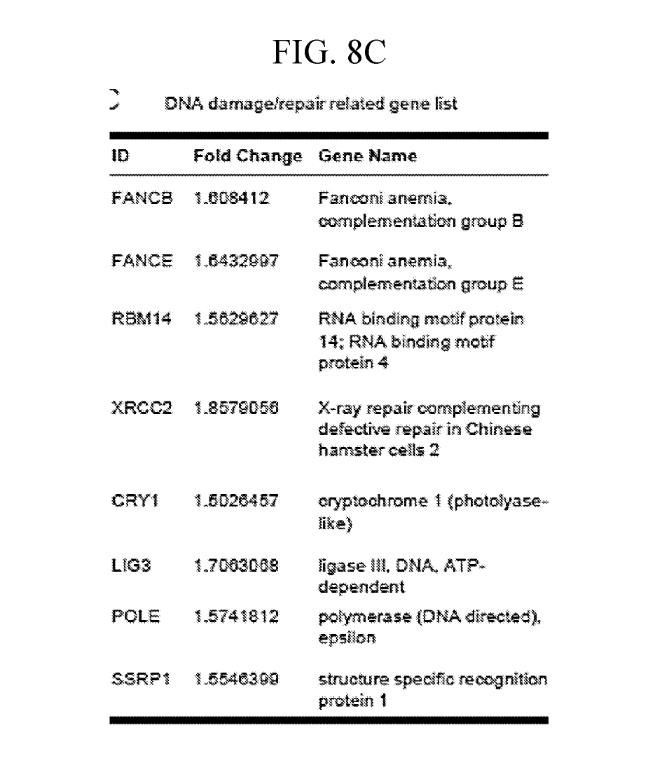

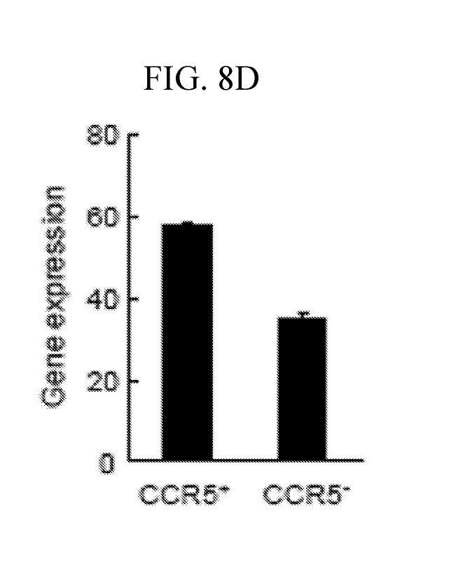

CCR5 induces DNA repair gene expression and function. In order to characterize the functional pathways regulated by CCR5 within the SUM159 basal breast cancer cells, the CCR5+ cells were separated by FACS sorting and subjected to microarray mRNA analysis. The KEGG pathway identified a subset of pathways induced in CCR5+ breast cancer cells, including pathways involved in DNA repair and response to DNA damage stimulus. The DNA repair and response to DNA damage stimulus included 8 genes (FIG. 8C). The DNA repair related genes involved members of most DNA repair pathways, including components of the Fanconi Anemia Pathway (FANCB, FANCE, NHEJ (RBM14), HRR (XRCC2), NER (POLE) and HOR (SSRP1). These changes in abundance were confirmed by QRT-PCR.

Together, these studies demonstrate that endogenous CCR5 within the breast cancer is sufficient to promote tumor metastasis and that CCR5 expression enhances properties of breast cancer stem cells associated with induction of DNA damage repair signaling. As the DNA repair pathways included the Fanconi anemia pathway, the homologous DNA repair, and the BER pathway, we conducted functional analyses comparing CCR5+ vs. CCR5− cells grade through FACS analysis. The DNA repair activity was enhanced in CCR5+ vs. CCR5− cells.

FIG. 4. CCR5⁺ population of SUM159 cells are enriched with breast cancer stem cells which showed by mammosphere formation assay. FIG. 4A FACS analysis of SUM159 cells demonstrating 5-10% of SUM159 cells are CCR5⁺. FIGS. 4B and 4C CCR5⁺ SUM159 cells formed more mammospheres than CCR5⁻ SUM159 cells. The typical photos of mammosphere derived from CCR5⁺ and CCR5⁻ SUM159 cells were showed in FIG. 4B and the Mean of the number of mammospheres formed per 1000 cells was showed in FIG. 4C. FIG. 4D SUM159 cells cultured in the condition which favored to differentiation (in the presence of 1% of DMSO or Normal DMEM media) have less CCR5⁺ cells.

FIG. 5. CCR5⁺ SUM159 cells are more tumorigenic than CCR5⁻ SUM159 cells. FIG. 5A Photos of photon flux from breast tumors of nude mice injected with CCR5+ vs. CCR5− breast cancer cells. An equal number of cells were injected in each animal. FIG. 5B Quantitation of photon-flux of tumors from mice at time 0 months and 4 months. FIG. 5C Size of tumors for individual mice by photon flux demonstrating the increased tumor mass of cells for CCR5+ vs. CCR5− cells, (1.94±0.94)×10⁸ vs (2.52±2.27)×10⁵, p<0.05 with Mann-Whitney Test.

FIG. 6. Overexpression of CCR5 in SUM159 breast cancer cells enhance their tumorigenecity. SUM159 cells stable transfected with either CCR5 or vector control were subcutaneously injected into the mice. Individual mouse tumors are shown. FIG. 6A Representative photon emission images of mice at 6 weeks. FIG. 6B Size of tumors for CCR5 re-expressing or vector control (CCR5−) animals during five week. Note log scale mean tumor volume detected by photon image is significantly different between CCR5⁺ vs. CCR5 vector control (2.24×10⁹±0.75×10⁹ vs 4.63×10⁵±1.49×10⁵, p<0.05 with student t-test).

FIG. 7. Endogenous CCR5 promotes lung metastases in mice. FIG. 7A Representative timed photon emission of mice injected with SUM159 cells treated with either control or Maraviroc (at 14 mg/kg mouse body, time as shown in weeks). FIG. 7B Mean±SEM of photon flux for 6 animals in control group and 7 animals in maraviroc treatment group shown in 5 weeks. Maraviroc treatment reduces mean lung tumor volume by 98% which detected by photon flux ((3.01±1.16)×10⁷ vs (1.05±0.44)×10⁸, p=0.063 with Mann-Whitney Test).

FIG. 8. CCR5⁺ cells within SUM159 activate DNA damage repair signaling pathways. FIG. 8A Microarray gene expression profiling of CCR5⁺ vs. CCR5⁻ cells separated from SUM159 breast cancer cells by FACS sorting. Changes in gene expression are shown by the calorimetric bar. FIG. 8B Gene-Ontology pathway analysis demonstrates distinct pathways induced or repressed in CCR5⁺ cells. The DNA repair and response to DNA damage pathways were 3.26 fold enrichment among up-regulated genes in CCR5⁺ vs CCR5⁻ cells (Additional pathways include actin, filament based processes, cytoskeleton, and alanyl+RNA amino acetylation). The response to unfolded proteins pathway was also induced. FIG. 8C the list of upregulated genes related to DNA repair/response to DNA damage pathways in CCR5⁺ SUM159 cells. FIG. 8D CCR5 expression in CCR5⁺ and CCR5⁻ cells demonstrates the efficiency of FACS sorting.

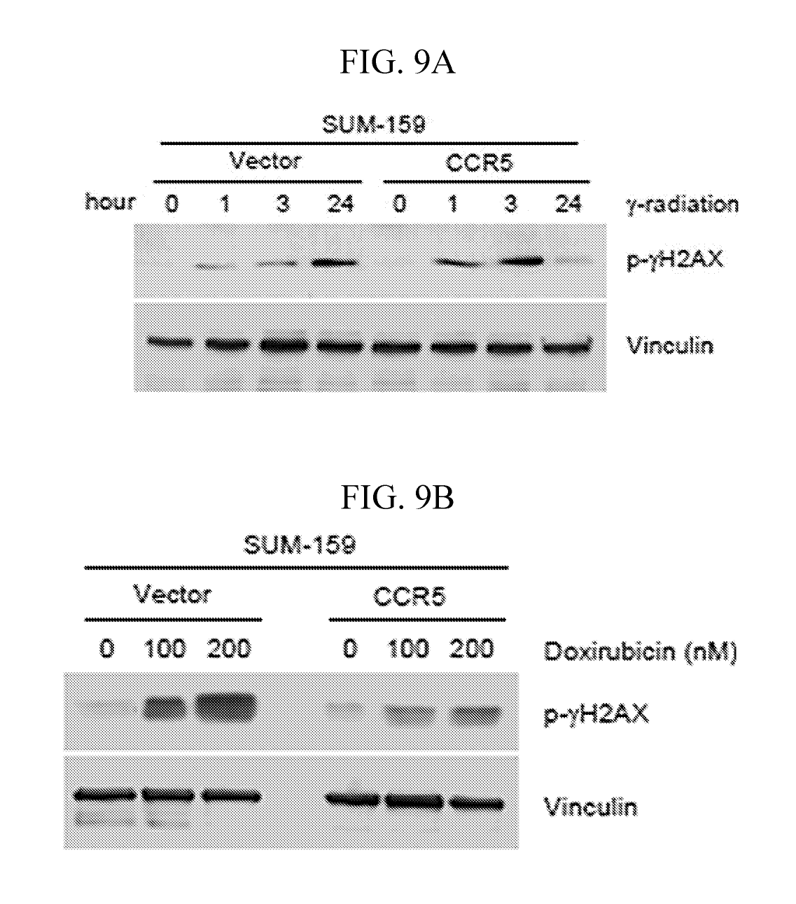

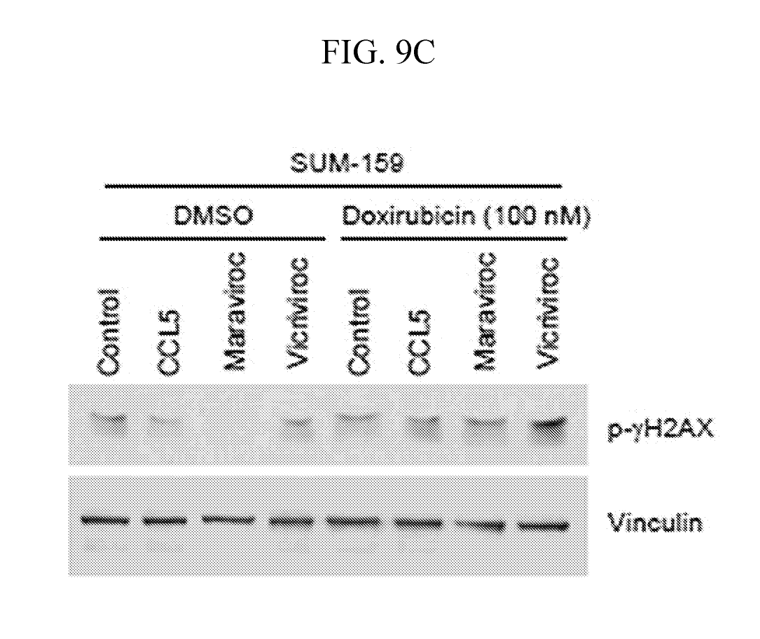

FIG. 9. CCR5 increased DNA damage repair in SUM159 breast cancer cells showed by Phospho-γH2AX Western-blot. FIG. 9A The CCR5 stable-transfected and vector-control SUM159 cells were illuminated with 6.5 Gy of γ-radiation. The samples were collected at 0.5, 3 and 24 hours after y-radiation. The recruitment of phosphor-γH2AX and the reduction of abundance were faster in CCR5-transfected cells than vector-control cells. FIG. 9B The CCR5 stable-transfected and vector-control SUM159 cells were treated with doxorubicin. DNA damage which showed by abundance of phospho-γH2AX was dramatically decreased in CCR5-transfected cells. FIG. 9C CCR5 antagonist increased DNA damage in SUM159 cells. SUM159 cells treated with CCR5 antagonist, Maraviroc and Vicriviroc, both at 100 μM. DNA damage was induced by doxorubicin treatment. There are more doxorubicin induced phospho-γH2AX in Marviroc and Vicriviroc treated groups.

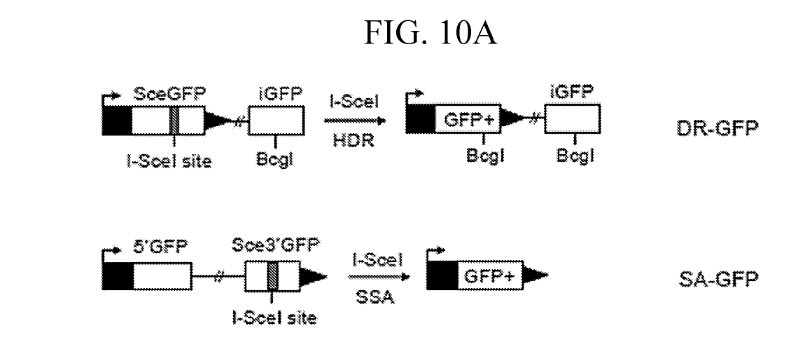

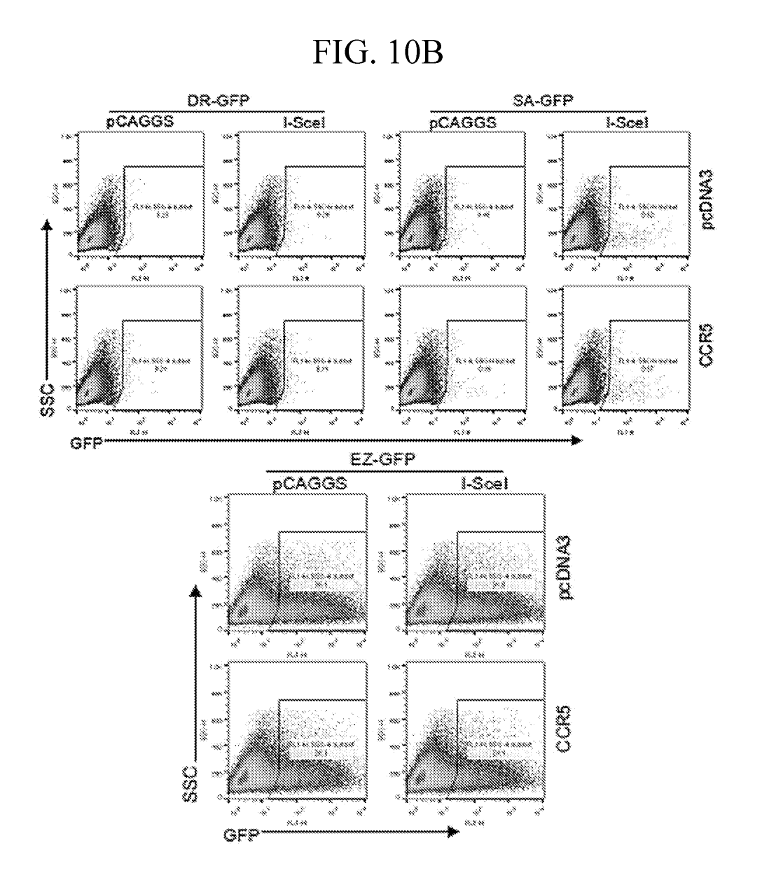

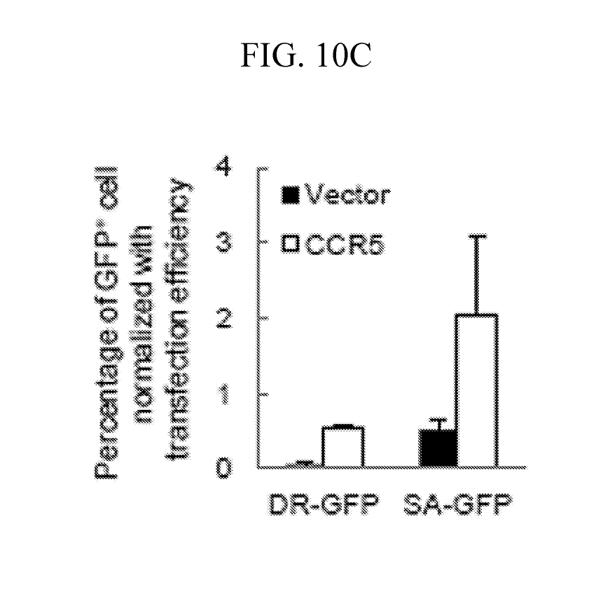

FIG. 10. DNA repair reporter assays conducted in SUM159 cells. FIG. 10A Reporters for homology-directed repair (HDR) and single strand annealing (SSA) of double-strand DNA breaking, DR-GFP for HDR and SA-GFP for SSA. FIG. 10B CCR5 increased both HDR and SSA activities. The cells were co-transfected with the plasmid encoding CCR5, plasmid encoding I-SceI and I-SceI based DNA repair reporter DR-GFP or SA-GFP. GFP+ cells which generated by HDR or SSA of I-SceI induced double-strand DNA were sorted by FACS. FIG. 10C the percentage of DR-GFP+ or SAGFP+ cells were calculated and normalized with transfection efficiency control EZ-GFP. DNA repair was quantitated. Representative example of the repair assays are shown. Compared with vector control, CCR5 increased both DR-GFP+ and SA-GFP+ cells.

All patents and publications cited herein are hereby fully incorporated by reference in their entirety. The citation of any publication is for its disclosure prior to the filing date and should not be construed as an admission that such publication is prior art or that the present invention is not entitled to antedate such publication by virtue of prior invention.

### CITATIONS

1. Velasco-Velazquez, M., Jiao, X., De La Fuente, M., Pestell, T. G., Ertel, A., Lisanti, M. P., and Pestell, R. G. CCR5 antagonist blocks metastasis of basal breast cancer cells. Cancer Res, 72: 3839-3850, 2012.

2. Sicoli, D., Jiao, X., Ju, X., Velasco-Velazquez, M., Ertel, A., Addya, S., Li, Z., Ando, S., Fatatis, A., Paudyal, B., Cristofanilli, M., Thakur, M., and Pestell, R. G. CCR5 receptor antagonists block metastasis to bone of Src-oncogene-transformed metastatic prostate cancer cell lines. Cancer Res: In Press, 2014.

3. Wu, K., Katiyar, S., Li, A., Liu, M., Ju, X., Popov, V. M., Jiao, X., Lisanti, M. P., Casola, A., and Pestell, R. G. Dachshund inhibits oncogene-induced breast cancer cellular migration and invasion through suppression of interleukin-8. Proc Natl Acad Sci USA, 105: 6924-6929, 2008.

4. Wu, K., Jiao, X., Li, Z., Katiyar, S., Casimiro, M. C., Yang, W., Zhang, Q., Willmarth, N. E., Chepelev, I., Crosariol, M., Wei, Z., Hu, J., Zhao, K., and Pestell, R. G. Cell fate determination factor Dachshund reprograms breast cancer stem cell function. J Biol Chem, 286: 2132-2142, 2011.

5. Bos, P. D., Zhang, X. H., Nadal, C., Shu, W., Gomis, R. R., Nguyen, D. X., Minn, A. J., van de Vijver, M. J., Gerald, W. L., Foekens, J. A., and Massague, J. Genes that mediate breast cancer metastasis to the brain. Nature, 459: 1005-1009, 2009.

6. Zhang, X. H., Jin, X., Malladi, S., Zou, Y., Wen, Y. H., Brogi, E., Smid, M., Foekens, J. A., and Massague, J. Selection of bone metastasis seeds by mesenchymal signals in the primary tumor stroma. Cell, 154: 1060-1073, 2013.

7. Velasco-Velazquez, M., Xolalpa, W., and Pestell, R. G. The potential to target CCL5/CCR5 in breast cancer. Expert Opin Ther Targets: 1-11, 2014.

8. Velasco-Velazquez, M. A., Homsi, N., De La Fuente, M., and Pestell, R. G. Breast cancer stem cells. Int J Biochem Cell Biol, 44: 573-577, 2012.

9. Velasco-Velazquez, M. A., Popov, V. M., Lisanti, M. P., and Pestell, R. G. The Role of Breast Cancer Stem Cells in Metastasis and Therapeutic Implications. Am J Pathol. 2011; 179(1):2-11. PMCID: 3123864.

10. Li, Z., Chen, K., Jiao, X., Wang, C., Willmarth, N. E., Casimiro, M. C., Li, W., Ju, X., K12. Camp, R. L., Chung, G. G., and Rimm, D. L. Automated subcellular localization and quantification of protein expression in tissue microarrays. Nat Med, 8: 1323-1327, 2002.

13. Dolled-Filhart, M., McCabe, A., Giltnane, J., Cregger, M., Camp, R. L., and Rimm, of beta-catenin expression in breast cancer shows decreased expression is associated with poor outcome. Cancer Res, 66: 5487-5494, 2006.

14. McCabe, A., Dolled-Filhart, M., Camp, R. L., and Rimm, D. L. Automated quantitative analysis (AQUA) of in situ protein expression, antibody concentration, and prognosis. J Natl Cancer Inst, 97: 1808-1815, 2005.

15. Peck, A. R., Witkiewicz, A. K., Liu, C., Stringer, G. A., Klimowicz, A. C., Pequignot, E., Freydin, B., Tran, T. H., Yang, N., Rosenberg, A. L., Hooke, J. A., Kovatich, A. J., Nevalainen, M. T., Shriver, C. D., Hyslop, T., Sauter, G., Rimm, D. L., Magliocco, A. M., and Rui, H. Loss of nuclear localized and tyrosine phosphorylated StatS in breast cancer predicts poor clinical outcome and increased risk of antiestrogen therapy failure. J Clin Oncol, 29: 2448-2458, 2011.

16. Sato, T., Tran, T. H., Peck, A. R., Girondo, M. A., Liu, C., Goodman, C. R., Neilson, L. M., Freydin, B., Chervoneva, I., Hyslop, T., Kovatich, A. J., Hooke, J. A., Shriver, C. D., Fuchs, S. Y., and Rui, H. Prolactin suppresses a progestin-induced CK5-positive cell population in luminal breast cancer through inhibition of progestin-driven BCL6 expression. Oncogene, 2013.

17. Sato, T., Tran, T. H., Peck, A. R.on, L. M., Liu, C., Brill, K. L., Rosenberg, A. L., Witkiewicz, A. K., and Rui, H. Prolactin inhibits BCL6 expression in breast cancer through a Stat5a-dependent mechanism. Cancer Res, 70: 1711-1721, 2010.

19. Yang, N., Liu, C., Peck, A. R., Girondo, M. A., Yanac, A. F., Tran, T. H., Utama, F. E., Tanaka, T., Freydin, B., Chervoneva, I., Hyslop, T., Kovatich, A. J., Hooke, J. A., Shriver, C. D., and Rui, H. Prolactin-Stat5 signaling in breast cancer is potently disrupted by acidosis within the tumor microenvironment. Breast Cancer Res, 15: R73, 2013.

20. LeBaron, M. J., Crismon, H. R., Utama, F. E., Neilson, L. M., Sultan, A. S., Johnson, K. J., Andersson, E. C., and Rui, H. Ultrahigh density microarrays of solid samples. Nat Methods, 2: 511-513, 2005.

21. Rui, H. and LeBaron, M. J. Creating tissue microarrays by cutting-edge matrix assembly. Expert Rev Med Devices, 2: 673-680, 2005.

22. LeBaron, M. J., Ahonen, T. J., Nevalainen, M. T., and Rui, H. In vivo response-based identification of direct hormone target cell populations using high-density tissue arrays. Endocrinology, 148: 989-1008, 2007.

23. Heagerty, P. J., Lumley, T., and Pepe, M. S. Time-dependent ROC curves for censored survival data and a diagnostic marker. Biometrics, 56: 337-344, 2000.

24. Chambless, L. E. and Diao, G. Estimation of time-dependent area under the ROC curve for long-term risk prediction. Stat Med, 25: 3474-3486, 2006.

25. Adams, D. L., Martin, S. S., Alpaugh, R. K., Charpentie, M., Tsai, S., Bergan, R. C., Ogden, I. M., Catalona, W., Chumsri, S., Tang, C. M., an111: 3514-3519, 2014.

26. Parkin D M, Fernandez L M. Use of statistics to assess the global burden of breast cancer. Breast J. 2006; 12 Suppl 1:S70-80.

27. Effects of chemotherapy and hormonal therapy for early breast cancer on recurrence and 15-year survival: an overview of the randomised trials. Lancet. 2005; 365(9472): 1687-717.

28. Meyers M O, Klauber-Demore N, Ollila D W, Amos K D, Moore D T, Drobish A A, Burrows E M, Dees E C, Carey L A. Impact of breast cancer molecular subtypes on locoregional recurrence in patients treated with neoadjuvant chemotherapy for locally advanced breast cancer. Ann Surg Oncol. 2011; 18(10):2851-7.

29. Kennecke H, Yerushalmi R, Woods R, Cheang M C, Voduc D, Speers C H, Nielsen T O, Gelmon K. Metastatic behavior of breast cancer subtypes. J Clin Oncol. 2010; 28(20):3271-7.

30. Luboshits G, Shina S, Kaplan O, Engelberg S, Nass D, Lifshitz-Mercer B, Chaitchik S, Keydar I, Ben-Baruch A. Elevated expression of the CC chemokine regulated on activation, normal T cell expressed and secreted (RANTES) in advanced breast carcinoma. Cancer Res. 1999; 59(18):4681-7.

31. Zhang Y, Yao F, Yao X, Yi C, Tan C, Wei L, Sun S. Role of CCL5 in invasion, proliferation and proportion of CD44+/CD24− phenotype of MCF-7 cells and correlation of CCL5 and CCR5 expression with breast cancer progression. Oncol Rep. 2009; 21(4):1113-21.

32. Al-Hajj M, Wicha M S, Benito-Hernandez A, Morrison S J, Clarke M F. Prospective identification of tumorigenic breast cancer cells. Proc Natl Acad Sci USA. 2003; 100(7):3983-8. PMCID: 153034.

33. Maugeri-Sacca M, Bartucci M, De Maria R. DNA damage repair pathways in cancer stem cells. Mol Cancer Ther. 2012; 11(8):1627-36.

34. Levy-Lahad E. Fanconi anemia and breast cancer susceptibility meet again. Nat Genet. 2010; 42(5):368-9.

35. D'Andrea A D. Susceptibility pathways in Fanconi's anemia and breast cancer. N Engl J Med. 2010; 362(20):1909-19. PMCID: 3069698.

36. Ponti D, Costa A, Zaffaroni N, Pratesi G, Petrangolini G, Coradini D, Pilotti S, Pierotti M A, Daidone M G. Isolation and in vitro propagation of tumorigenic breast cancer cells with stem/progenitor cell properties. Cancer Res. 2005; 65(13):5506-11.

What is claimed is:

1. A method of treating a subject with cancer, the method comprising:
Obtaining a sample from said patient detecting the presence of CCR5 on circulating tumor cells in said sample, wherein detecting the presence of CCR5 on circulating tumor cells comprises contacting an antibody that binds to CCR5 with a population of circulating tumor cells and detecting the bound antibody to determine the presence of CCR5 on circulating tumor cells; and
administering to the subject with CCR5 on the circulating tumors cells a CCR5 inhibitor to treat the cancer.

2. The method of claim 1, wherein detecting the presence of CCR5 on circulating tumor cells comprises using flow cytometry to detect CCR5 on circulating tumor cells.

3. (canceled)

4. (canceled)

5. The method of claim 1, wherein the circulating tumor cell is a breast cancer circulating tumor cell.

6. The method of claim 1, wherein the circulating tumor cell is a prostate cancer circulating tumor cell.

7. The method of claim 1, wherein the CCR5 inhibitor is 4,4-difluoro-N-[(1S)-3-[(1R,5S)-3-(3-methyl-5-propan-2-yl-1,2,4-triazol-4-yl)-8-azabicyclo[3.2.1]octan-8-yl]-1-phenylpropyl]cyclohexane-1-carboxamide; N-(1S)-3-3-(3-Isopropyl-5-methyl-4H-1,2,4-triazol-4-yl)-exo-8-azabicyclo[3.2.1]oct-8-yl-1-phenylpropylcyclobutanecarboxamide; N-(1S)-3-3-(3-Isopropyl-5-methyl-4H-1,2,4-triazol-4-yl)-exo-8-azabicyclo[3.2.1]oct-8-yl-1-phenylpropylcyclopentanecarboxamide; N-(1S)-3-3-(3-Isopropyl-5-methyl-4H-1,2,4-triazol-4-yl)-exo-8-azabicyclo[3.2.1]oct-8-yl-1-phenyl propyl-4,4,4-trifluorobutanamide; N-(1S)-3-3-(3-Isopropyl-5-methyl-4H-1,2,4-triazol-4-yl)-exo-8-azabicyclo[3.2.1]oct-8-yl-1-phenylpropyl-4,4-difluorocyclohexanecarboxamide; N-(1S)-3-3-(3-Isopropyl-5-methyl-4H-1,2,4-triazol-4-yl)-exo-8-azabicyclo[3.2.1]oct-8-yl-1-(3-fluorophenyl)propyl-4,4-difluorocyclohexanecarboxamide; and pharmaceutically acceptable salts or solvates thereof.

8. The method of claim 7, wherein the CCR5 inhibitor is maraviroc or vicriviroc.

9. (canceled)

10. The method of claim 1, wherein the sample is a blood sample or a urine sample.

11. (canceled)

12. A method of identifying and treating a cancer in a subject as susceptible to a CCR5 inhibitor, the method comprising:
obtaining a sample from the subject;
detecting the presence of CCR5 on circulating tumor cells in the subject's sample; wherein the presence is detected by of CCR5 is detected from using flow cytometry to detect CCR5 on circulating tumor cells, contacting an antibody that binds to CCR5 with a population of circulating tumor cells and detecting the bound antibody to determine the presence of CCR5 on circulating tumor cells, or performing an ELISA to detect the presence of CCR5 on circulating tumor cells;
identifying the cancer as susceptible to a CCR5 inhibitor when CCR5 is found to be present on the circulating tumor cells; and
administering to the identified subject a CCR5 inhibitor to treat the cancer.

13. (canceled)

14. (canceled)

15. (canceled)

16. The method of claim 12, wherein the cancer is breast cancer.

17. The method of claim 12, wherein the cancer is prostate cancer.

18. A method of treating a subject with cancer, the method comprising:
obtaining a sample from said subject; detecting the presence of CCR5 on circulating tumor cells in a subject's sample; and
administering to the subject with CCR5 on the circulating tumors cells a CCR5 inhibitor and a DNA damaging agent to treat the cancer.

19. The method of claim 18, wherein detecting the presence of CCR5 on circulating tumor cells comprises using flow cytometry to detect CCR5 on circulating tumor cells.

20. The method of claim 18, wherein detecting the presence of CCR5 on circulating tumor cells comprises contacting an antibody that binds to CCR5 with a population of circulating tumor cells and detecting the bound antibody to determine the presence of CCR5 on circulating tumor cells.

21. The method of claim 18, wherein detecting the presence of CCR5 on circulating tumor cells comprises performing an ELISA to detect the presence of CCR5 on circulating tumor cells.

22. The method of claim 18, wherein the CCR5 inhibitor is maraviroc or vicriviroc.

23. The method of claim 18, wherein the DNA damaging agent is an alkylating agent, intercalating agents, or a polymerase inhibitor.

24. The method of claim 18, wherein the DNA damaging agent is busulfan, bendamustine, carboplatin, carmustine, chlorambucil, cisplatin, cyclophosphamide, dacarbazine, daunorubicin, decitabine, doxorubicin, epirubicin, etoposide, idarubicin, ifosfamide, irinotecan, lomustine, mechlorethamine, melphalan, mitomycin C, mitoxantrone, oxaliplatin, temozolomide, 5-fluorouracil, paclitaxel, topotecan, or any combination thereof.

25. The method of claim 18, wherein the DNA damaging agent is a PARP inhibitor.

26. The method of claim 18, wherein the cancer is breast or prostate cancer.

27-50. (canceled)
